(19) United States
(12) Patent Application Publication
(10) Pub. No.: US 2026/0133155 A1
(43) Pub. Date: May 14, 2026

(54) INTEGRATED SOIL SENSOR FOR MEASURING NUTRIENT IONS AT VARYING DEPTHS

(71) Applicant: BOARD OF REGENTS, THE UNIVERSITY OF TEXAS SYSTEM, Austin, TX (US)

(72) Inventor: Shawana TABASSUM, Tyler, TX (US)

(73) Assignee: BOARD OF REGENTS, THE UNIVERSITY OF TEXAS SYSTEM, Austin, TX (US)

(21) Appl. No.: 19/324,841

(22) Filed: Sep. 10, 2025

Related U.S. Application Data
(60) Provisional application No. 63/718,981, filed on Nov. 11, 2024.

(51) Int. Cl. G01N 27/333; G01N 27/416; G01N 33/24

(52) U.S. Cl. CPC G01N 27/333; G01N 27/4166; G01N 33/24

(57) ABSTRACT

A real-time monitoring system for in situ soil nitrate concentration measurement comprising at least one 3D-printed and chemically functionalized potentiometric soil sensor for measuring nitrate levels and methods of making the real-time monitoring system. The real-time monitoring system is capable of providing multi-depth nitrate concentration measurements and continuous measurements over time.

### CROSS REFERENCE TO RELATED APPLICATIONS

This application claims priority benefit to U.S. Provisional Application No. 63/718,981, filed Nov. 11, 2024, the disclosure of which is incorporated herein by reference in its entirety.

### STATEMENT REGARDING FEDERALLY SPONSORED RESEARCH OR DEVELOPMENT

This invention was made with Federal government support under USDA-NIFA 2023-67021-40548 awarded by the United States Department of Agriculture. The government has certain rights in the invention.

### COLOR DRAWINGS

The patent or application file contains at least one drawing executed in color. Copies of this patent or patent application publication with color drawing(s) will be provided by the Office upon request and payment of the necessary fee.

### TECHNICAL FIELD

The present disclosure relates generally to the agricultural field, and more specifically to a probe comprising an array of 3D-printed and chemically functionalized potentiometric soil sensors for measuring nitrate levels at different soil depths.

### BACKGROUND

The rapid increase in food demand puts immense pressure on our soil. However, less than 50% of synthetic nitrogen fertilizer applied to US soils is absorbed by crops, with the rest leaching as nitrate and contaminating surface and groundwater. Nitrogen fertilizer is converted to nitrate through microbial processes in the soil, where bacteria oxidize ammonium to nitrite and then to nitrate (NO₃⁻) in a process called nitrification. This process is essential for plant nutrition but must be carefully managed to prevent water pollution and greenhouse gas emissions (primarily N₂O). Therefore, measuring nitrate in soil is crucial for nutrient management, environmental protection, and optimized soil chemistry.

Various techniques are known for monitoring the nitrate level in soil. Typically, laboratory-based methods of analysis for measuring soil nitrogen are used. These methods include mass spectrometry, ultraviolet-visible spectrophotometry, ion chromatography, and chemiluminescence. These procedures require taking one or more soil samples from different depths to be analyzed and transporting them to a suitable laboratory where they are processed and analyzed. This analysis involve complex instrumentation, are high cost and require labor-intensive procedures. Thereafter, a corresponding analysis report is prepared and sent back to the farmer. As a rule, the time between the taking of the sample and the notification of the analysis result takes at least several days and up to several weeks during times of high demand. Although the methods which are used in such laboratory analyses are very accurate, they cannot be used in situ, either because the technical equipment which is required for this purpose is not mobile, or because the analysis requires standardized environmental conditions that can only be achieved in a laboratory. Additionally, laboratory-based methods of analysis lack real-time and spatiotemporal information. Due to its high cost, farmers typically test their soil only once every two to five years.

As an alternative to soil analysis in a laboratory, some methods for in situ or semi in situ analysis of soil are currently available on the market. However, these processes for in-field monitoring lack the ability to multiplex in a single probe; lack wireless data transmission capabilities; require frequent calibration and maintenance; encounter signal drift and decline in sensitivity over time. Additionally, various electrochemical sensors, including amperometric, potentiometric, and optical types, have shown significant potential for nitrate measurements. However, these sensors still face limitations in continuous and long-term in situ measurement at varying depths and moisture levels. These sensors also involve significant costs, particularly when several sensors need to be deployed in the field to achieve high-resolution spatiotemporal monitoring.

Thus, there is an unmet need for cost-effective sensing methods that offer efficient spatiotemporal analysis of nitrogen in the field.

### SUMMARY OF THE INVENTION

The present disclosure includes an array of 3D-printed and chemically functionalized potentiometric soil sensor arrays for measuring nitrate levels at different soil depths. In one aspect, this probe includes an array of three potentiometric soil sensors on a single real-time monitoring system to provide highly localized information on nitrate levels at three depths inside the soil. Each potentiometric soil sensor has one working electrode (WE) and one reference electrode (RE). The working electrode is coated with an ion-to-electron transduction layer followed by an ion-selective membrane (ISM) for selective detection of nitrate. The real-time monitoring system is interfaced with a data acquisition circuit that includes an analog front end (AFE), a microcontroller unit (MCU), and a data transmission circuit. The real-time monitoring system of the present disclosure can be used to evaluate the nitrogen content in a variety of plant species, including corn, cowpea, and tomato. In some aspects, data collected can be collected by the real-time monitoring system from sensors at different soil depths, including data collected from varying root lengths, root distribution, and nitrate uptake among different plant species, which are useful for agricultural purposes.

In one aspect, the present disclosure includes a potentiometric soil sensor comprising: (i) a substrate; and (ii) a nitrate sensor comprising a nitrate ion-selective membrane (ISM) disposed on the substrate; wherein the nitrate sensor comprises at least one working electrode (WE) and at least one reference electrode (RE); wherein the WE and RE each comprise one or more conductive layers, and wherein the WE and RE are disposed on a surface of the substrate.

In one aspect, the present disclosure includes a method for making at least one potentiometric soil sensor comprising the steps of:
a. 3D printing a substrate including at least two grooves on a front surface transversing from one end of the substrate to the other end of the substrate along a vertical direction;
b. disposing a WE comprising an active region and a connection region and a RE comprising an active region and a connection region into the two grooves on the front surface of the substrate.

In some aspects, the method comprises the steps of:
a. 3D printing a substrate including at least two grooves on a front surface transversing from one end of the substrate to the other end of the substrate along a vertical direction;
b. disposing a working electrode (WE) comprising an active region and a connection region and a reference electrode (RE) comprising an active region and a connection region into the two grooves on the front surface of the substrate;
c. brush coating the WE and the RE with carbon (C) ink;
d. brush coating silver or silver chloride (Ag/AgCl) ink on the C ink-coated RE of (c);
e. drop casting a nanocomposite on the active region of the C ink-coated WE of (c);
f. drop casting ISM on the nanocomposite-coated active region of WE of (e)
g. spray depositing single-walled carbon nanotubes (SWCNT) on top of the Ag/AgCl-coated RE of (d);
h. drop casting of RE membrane on top of the SWCNT coated RE of (g); and
i. forming a passivation layer on the connection region of the WE and the RE. Optionally, any one or more of the foregoing steps may be omitted or performed in a different order.

In one aspect, the present disclosure includes a probe comprising the potentiometric soil sensor of the disclosure. In some aspects, the probe comprises an array of the potentiometric soil sensors.

In one aspect, the present disclosure includes a method for making a real-time monitoring system for in situ soil nitrate concentration analysis comprising:
a. making at least one potentiometric soil sensor comprising the steps of: i. 3D printing a substrate including at least two grooves on a front surface and two grooves in the back surface transversing from one end of the substrate to the other end of the substrate along a vertical direction; ii. inserting a WE comprising an active region and a connection region and a RE comprising an active region and a connection region into the grooves of the substrate; iii. brush coating the WE and the RE with C ink; iv. brush coating Ag/AgCl ink on the C ink-coated RE of (iii); v. drop casting a nanocomposite on the active region of the C ink-coated WE of (iii); vi. drop casting ISM on the nanocomposite-coated active region of WE of (v); vii. spray depositing SWCNTs on top of the Ag/AgCl-coated RE of (iv); viii. drop casting of RE membrane on top of the SWCNT coated RE of (vii); ix. forming a passivation layer on the connection region of the WE and the RE; and
b. making at least one connector; and
c. mechanically attaching the least one potentiometric soil sensor to at least one connector. Optionally, any one or more of the foregoing steps may be omitted or performed in a different order. In some aspects, the ink may be substituted with a different ink.

In one aspect, the present disclosure includes a method of selectively measuring nitrate in soil, comprising: inserting a probe comprising at least one potentiometric soil sensor into a soil; generating an electrical potential between a reference electrode (RE) and a working electrode (WE), wherein the potential is based on nitrate levels in the soil; producing an electrical signal in response to the electrical potential between the RE and the WE, wherein said electrical signal indicates the soil nitrate concentration; transmitting the electrical signal to a data acquisition circuit to apply a calibration operation; determining the soil nitrate concentration based on the calibration operation.

The present disclosure provides cost-effective sensing methods that offer efficient spatiotemporal analysis of nitrogen in the field. This information is crucial for optimizing nitrogen application practices. The low-cost sensor would enable large-scale implementation in the field and the sensors can be scaled for use across larger regions. For these reasons, wireless, multiplexed, and low-cost sensors for use in collecting real-time data on nitrogen dynamics in the soil are an important advance in the field.

Each of the aspects of the present disclosure can encompass various elements of the present disclosure. It is, therefore, anticipated that each of the aspects of the present disclosure involving any one element or combinations of elements can be included in each aspect of the present disclosure. This disclosure is not limited in its application to the details of construction and the arrangement of components set forth in the following detailed description or illustrated in the drawings.

### BRIEF DESCRIPTION OF THE DRAWINGS

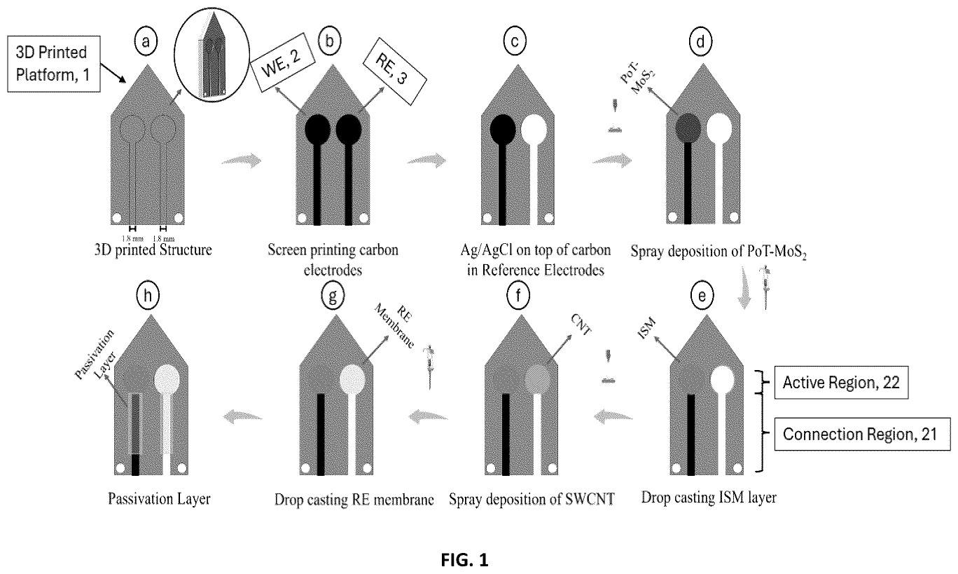

FIG. 1 shows the potentiometric soil sensor fabrication process including: (a) a substrate (“3-D printed platform”) (1); (b) brush coating of carbon ink on both the WE (2) and RE (3); (c) brush coating of Ag/AgCl ink on the carbon ink-coated RE; (d) spray deposition of the nanocomposite (“PoT-MoS₂”) on the WE; (e) drop casting of nitrate ISM on top of the nanocomposite layer; (f) spray deposition of SWCNT on top of Ag/AgCl-coated RE; (g) drop casting of RE membrane atop SWCNT to prevent chloride leaching; and (h) formation of a passivation layer on the connection region (21) without coating the active region (22).

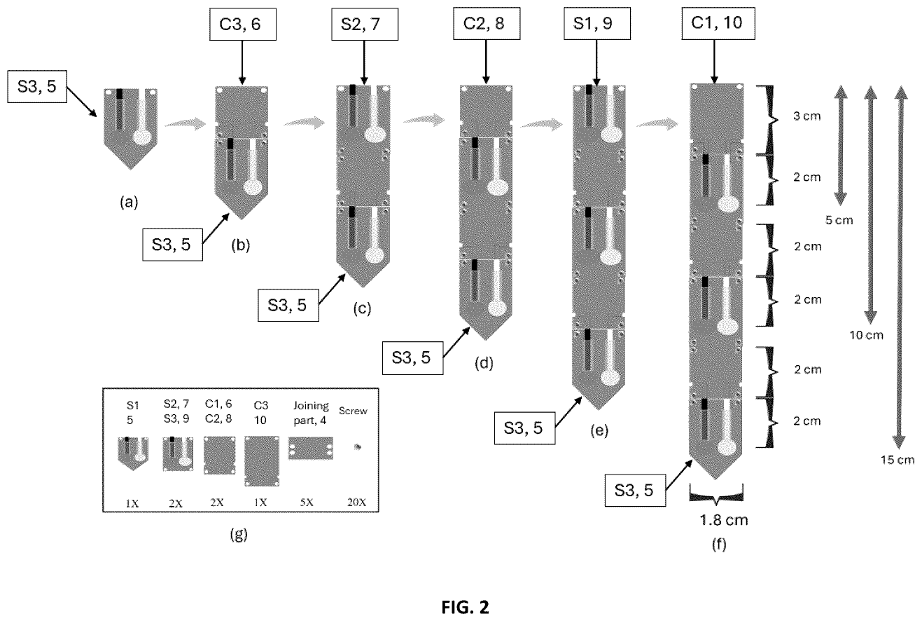

FIG. 2 shows the potentiometric soil sensor array integration process of producing one embodiment of the real-time monitoring system including: (a) sensor designated for a depth of 15 cm (S3, 5); (b) the 15-cm sensor (S3, 5) attached to a 2 cm connector (C3, 6); (c) attaching the sensor intended for 10 cm depth (S2, 7); (d) joining another 2 cm connector (C2, 8); (e) attaching the sensor intended for 5 cm depth (S1, 9); (f) joining a 3 cm connector (C1, 10) to form the final 3-sensor real-time monitoring system; and (g) total number of each part used to assemble the real-time monitoring system, wherein each connection between a connector and a sensor is attached using a joining part (4) and screws.

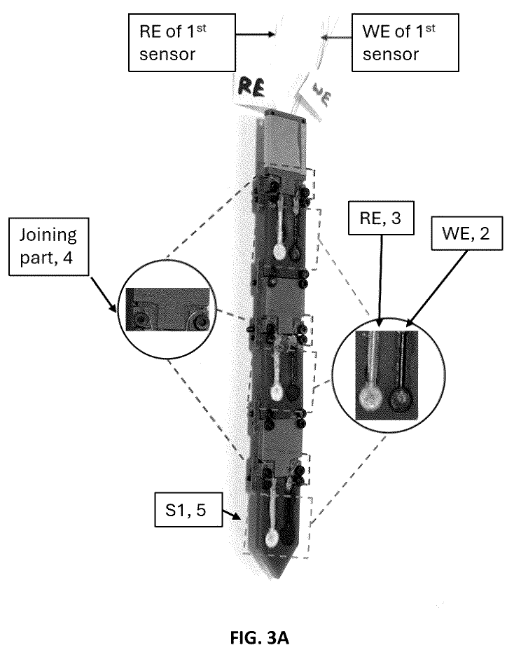

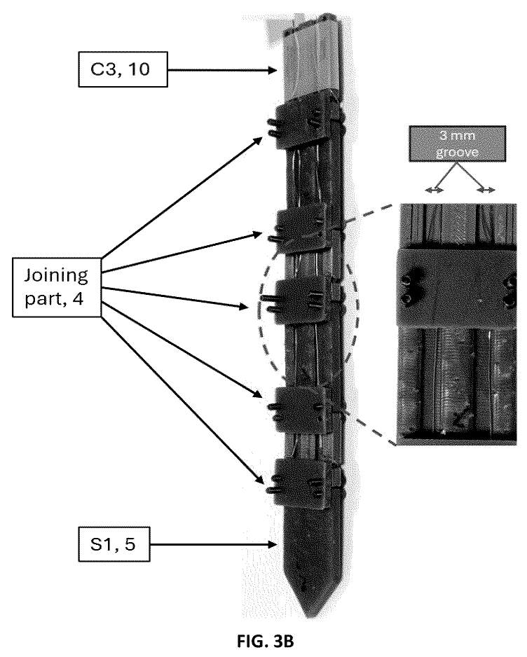

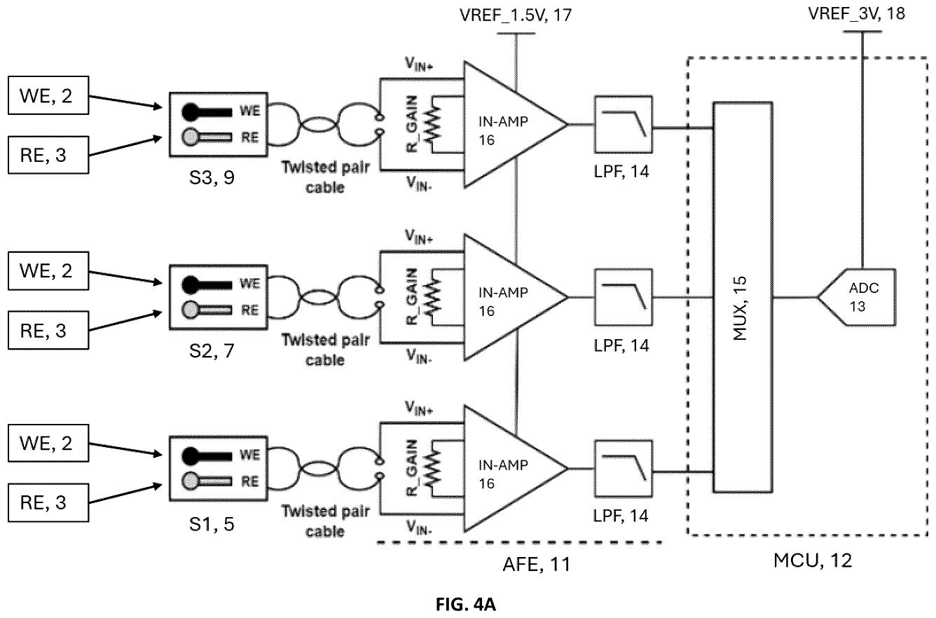

FIG. 3A shows the front view of the final structure following the assembly of the 3D printed parts, including the joining part (4) with 0.6 mm deep grooves and the insets of the RE (3) and WE (2) sensors. Each sensor features two 0.6 mm deep grooves for WE and RE. Each connector has two 0.6 mm deep grooves on the front to route the wires, while the rear surface of the sensor array also has grooves to pass the wires through. Each of the three sensors has two wires for the WE and RE, making a total of six wires.

FIG. 3B shows the back view of the final structure following the assembly of the 3D printed parts, showing the bottom sensor (S1, 5) having cone tip configured to penetrate a soil, 3 connectors mechanically attached to 3 sensors by joining parts (4) and wires of the RE and WE routed through two 3 mm wide grooves at the back of the structure, which accommodated the 0.5 mm diameter wires.

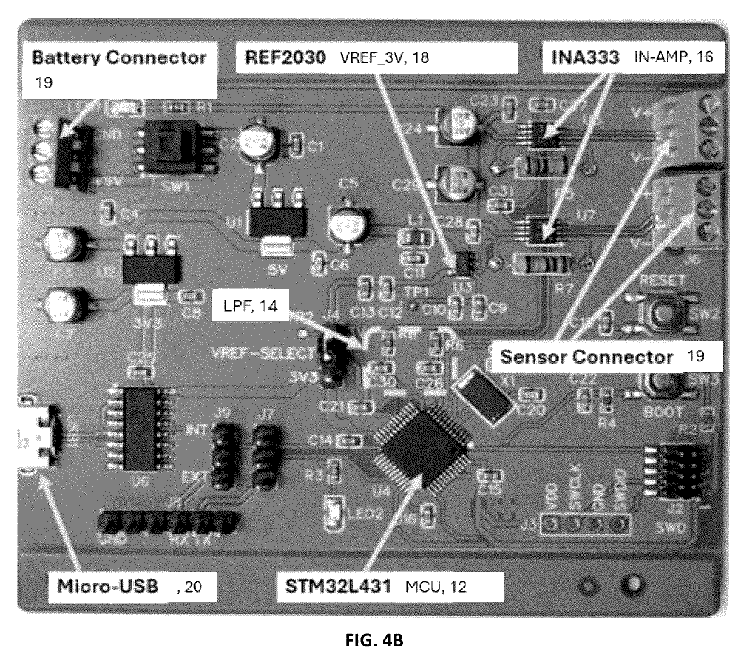

FIG. 4A shows a schematic diagram of the data acquisition circuit having three sets of WE (2) and RE (3) disposed in S3 (9), S2 (7) and S1 (5), each sensor connected to a instrumentation amplifier (IN-AMP, 16), the three IN-AMPs connected to a single 1.5V voltage reference (17) and each connected to a low pass filter (LPF, 14), each LPF attached to a single multiplexer (MUX, 15), the MUX connected to an analogue to digital converter (ADC, 13), the ADC connected to a 3V voltage reference (VREF_3V, 18); wherein the active electromagnetic interference (EMI) filter (AEF, 11) comprises the IN-AMP and LPF and the microcontroller unit (MCU, 12) comprises the MUX and ADC.

FIG. 4B shows an image of the data acquisition circuit including a battery connector (19), a VREF_3V (18), an IN-AMP (16), a LPF (14), a sensor connector (19), a Micro-USB (20) and a MCU (12).

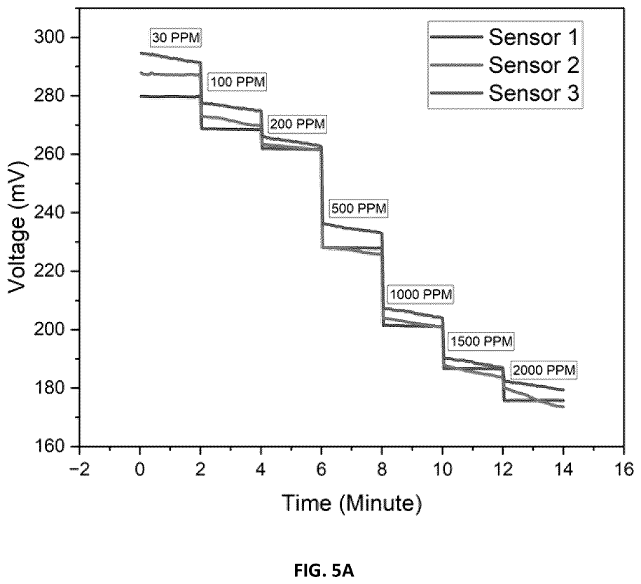

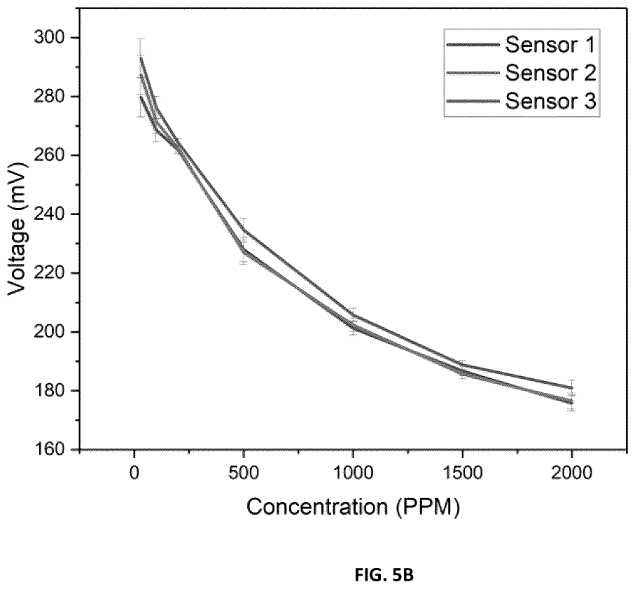

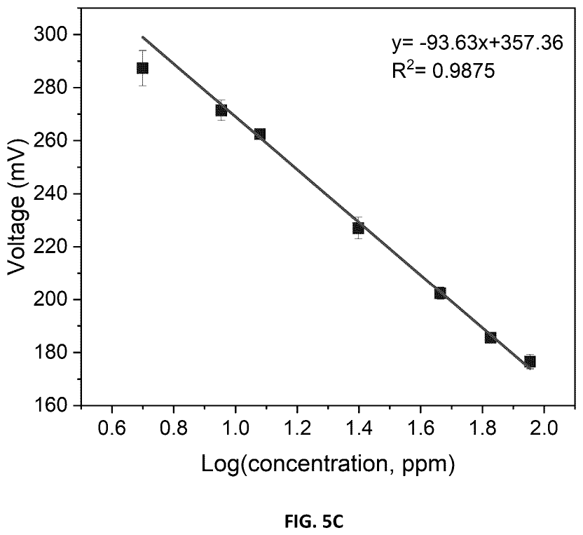

FIG. 5A shows the data of dynamic voltage response collected from three identical sensors.

FIG. 5B shows a calibration graph of voltage values versus nitrate concentrations collected from three identical sensors.

FIG. 5C shows a calibration graph of voltage response vs. logarithm of the nitrate concentration in ppm.

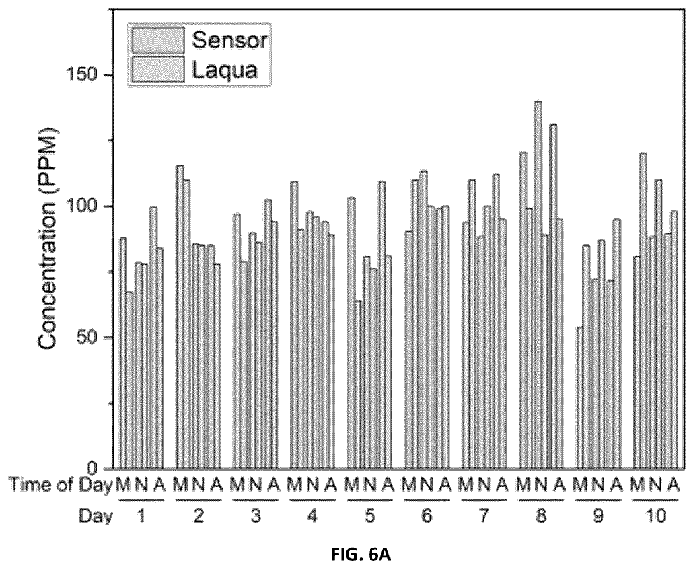

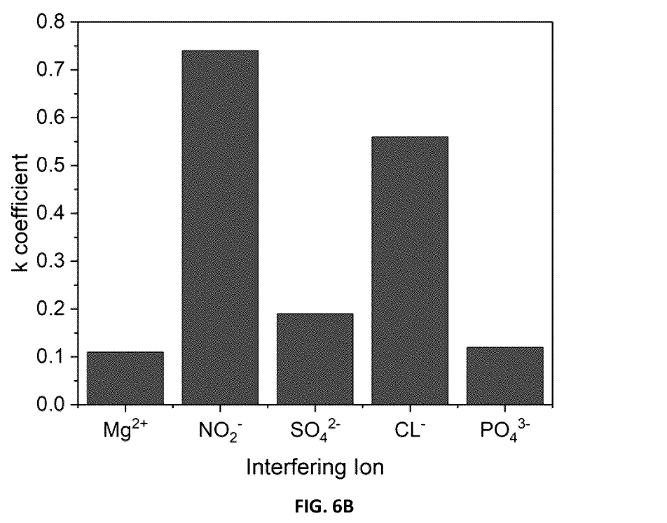

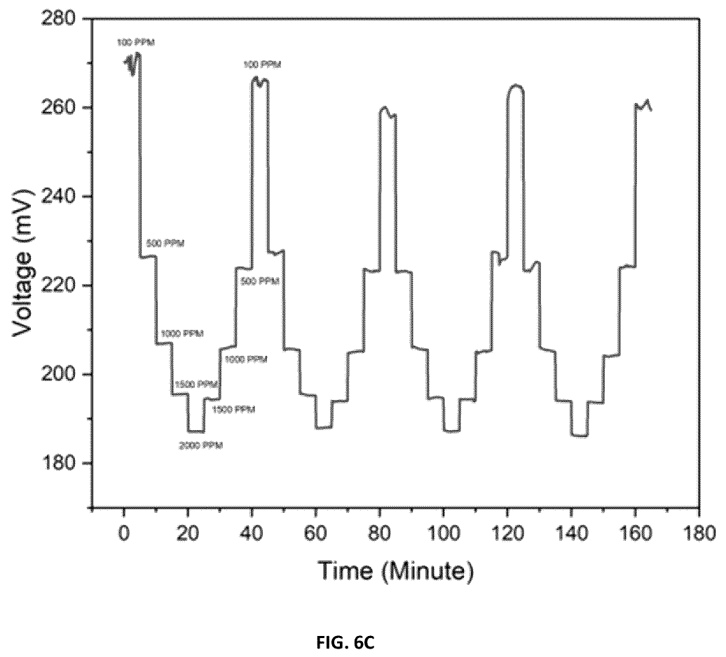

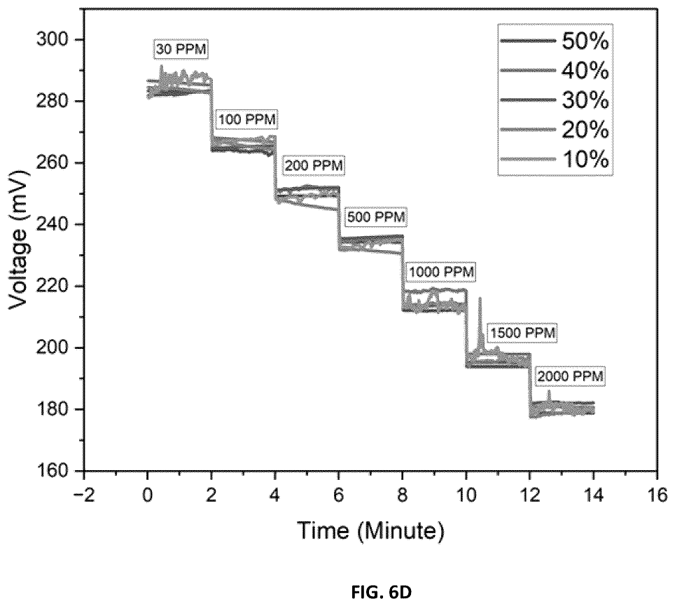

FIG. 6A shows the results of a 12-day longevity test with the sensor readings collected from a potentiometric soil sensor array of the disclosure validated against a commercially available sensor.

FIG. 6B shows the results of a selectivity test of the sensors of a potentiometric soil sensor array of the disclosure for nitrate concentration in soil containing 1000 ppm of each of Mg²⁺, NO²⁻, SO₄²⁻, Cl⁻, PO₄²⁻, and NO²⁻ with the sensor readings collected from a potentiometric soil sensor array of the disclosure validated against a commercial LaquaTwin sensor.

FIG. 6C shows the results of a selectivity test of the sensors of the disclosure in the presence of interfering ions, with the sensor readings collected from a potentiometric soil sensor array of the disclosure validated against a commercial LaquaTwin sensor.

FIG. 6D shows a calibration plot of the sensor at varying soil moisture levels, with the sensor readings collected from a potentiometric soil sensor array of the disclosure validated against a commercial LaquaTwin sensor.

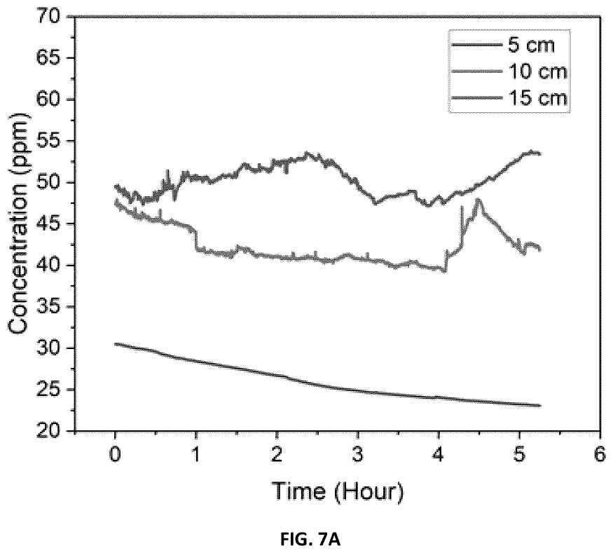

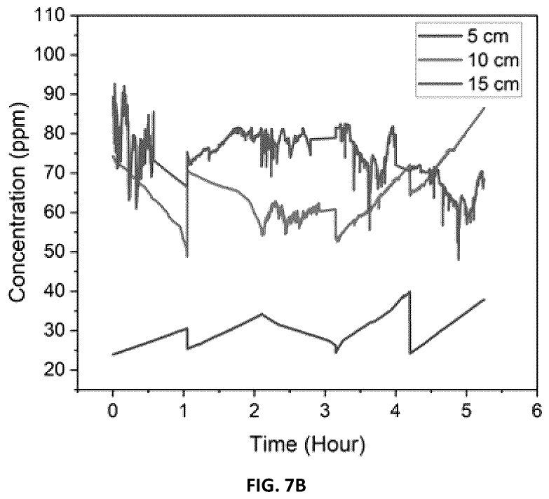

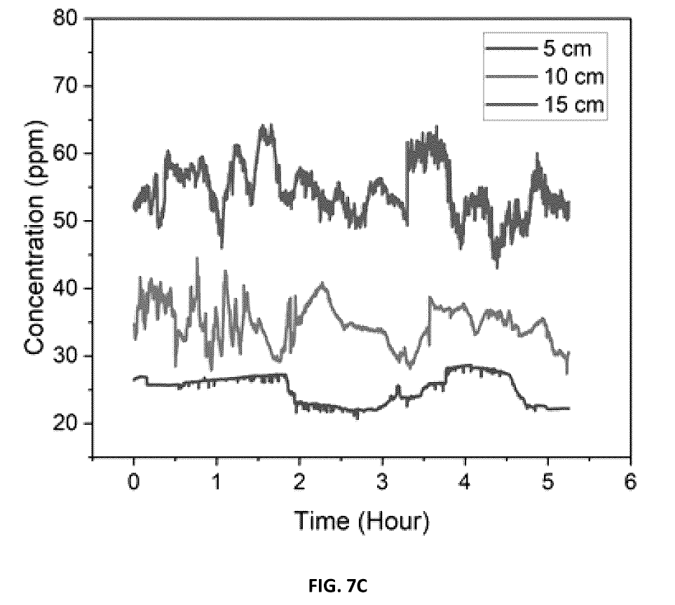

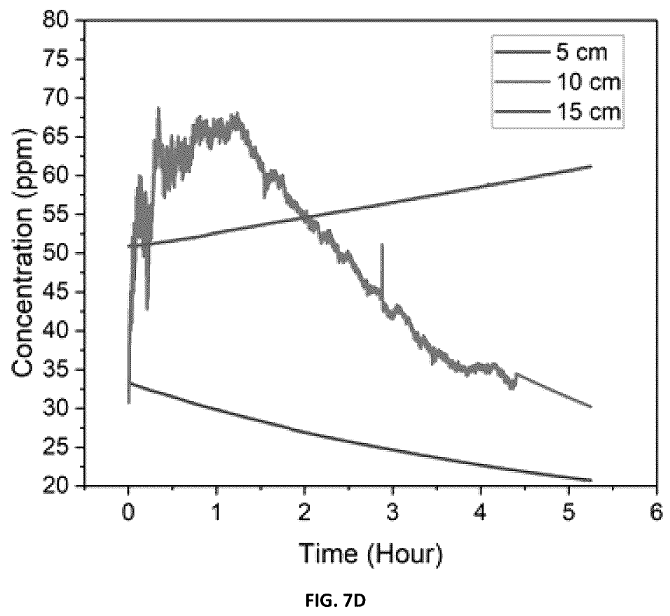

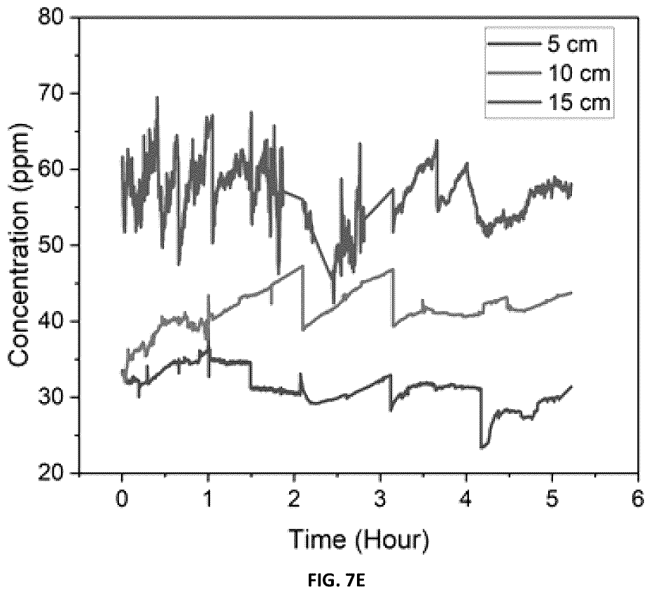

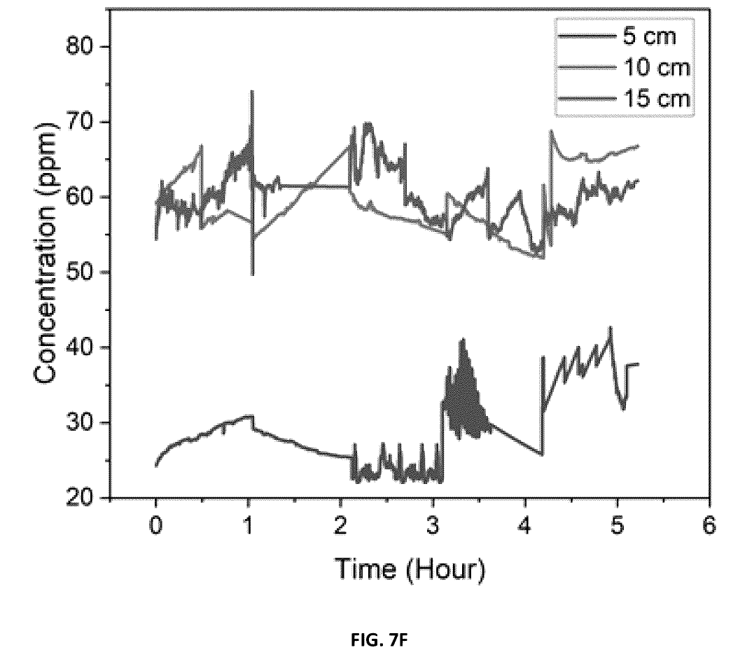

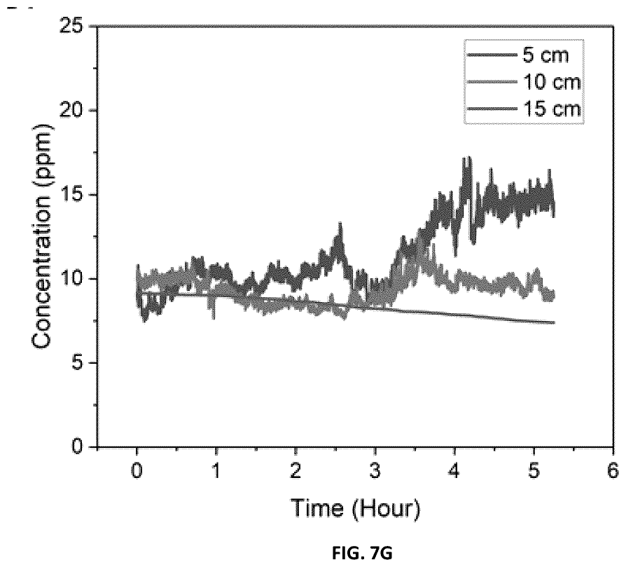

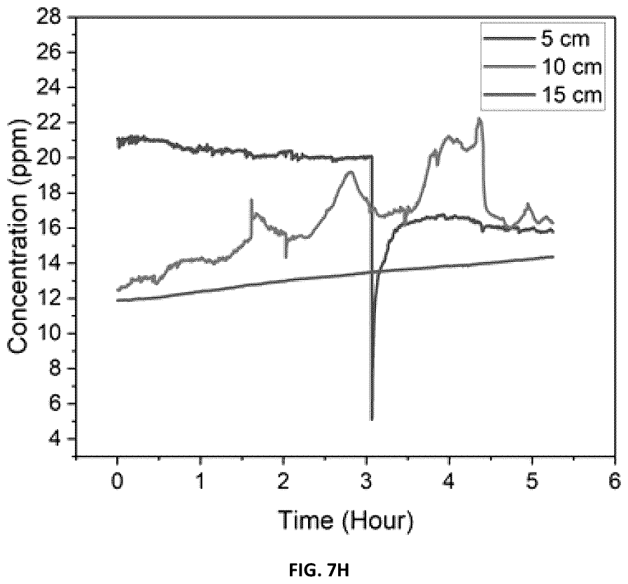

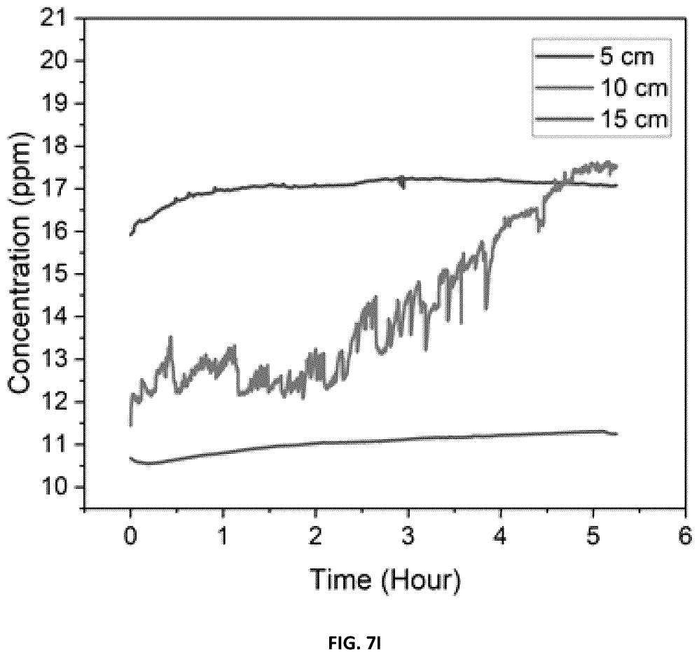

FIG. 7A shows dynamic nitrate measurements over a 5-hour period at varying moisture levels and different soil depths in Corn Bare soil.

FIG. 7B shows dynamic nitrate measurements over a 5-hour period at varying moisture levels and different soil depths in Corn Plant 1 soil.

FIG. 7C shows dynamic nitrate measurements over a 5-hour period at varying moisture levels and different soil depths in Corn Plant 2 soil.

FIG. 7D shows dynamic nitrate measurements over a 5-hour period at varying moisture levels and different soil depths in Cowpea Bare soil.

FIG. 7E shows dynamic nitrate measurements over a 5-hour period at varying moisture levels and different soil depths in Cowpea Plant 1 soil.

FIG. 7F shows dynamic nitrate measurements over a 5-hour period at varying moisture levels and different soil depths in Cowpea Plant 2 soil.

FIG. 7G shows dynamic nitrate measurements over a 5-hour period at varying moisture levels and different soil depths in Tomato Bare soil.

FIG. 7H shows dynamic nitrate measurements over a 5-hour period at varying moisture levels and different soil depths in Tomato Plant 1 soil.

FIG. 7I shows dynamic nitrate measurements over a 5-hour period at varying moisture levels and different soil depths in Tomato Plant 2 soil.

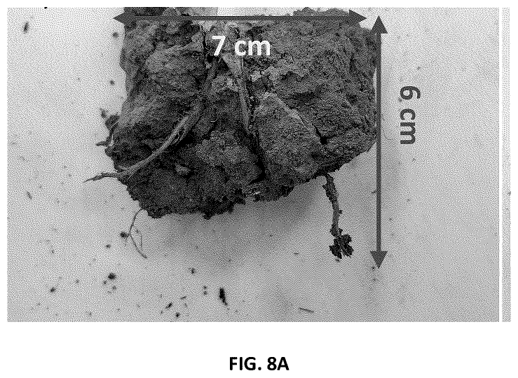

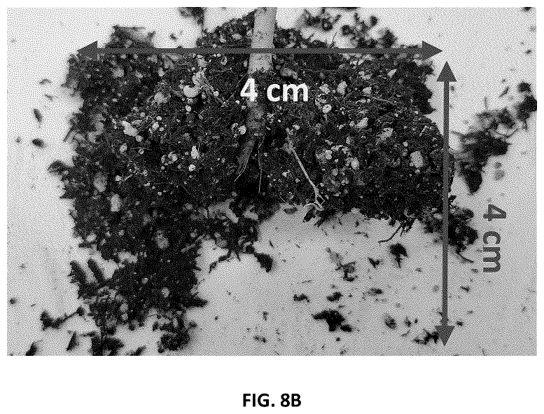

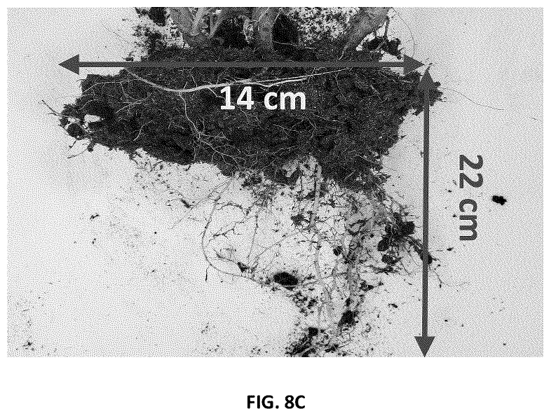

FIG. 8A shows lateral and vertical extension of Corn root with a 7 cm lateral stretch and a 6 cm vertical stretch.

FIG. 8B shows lateral and vertical extension of Cowpea root with a 4 cm lateral and 4 cm vertical stretch.

FIG. 8C shows lateral and vertical extension of Tomato root with a 14 cm lateral stretch and a 22 cm vertical stretch.

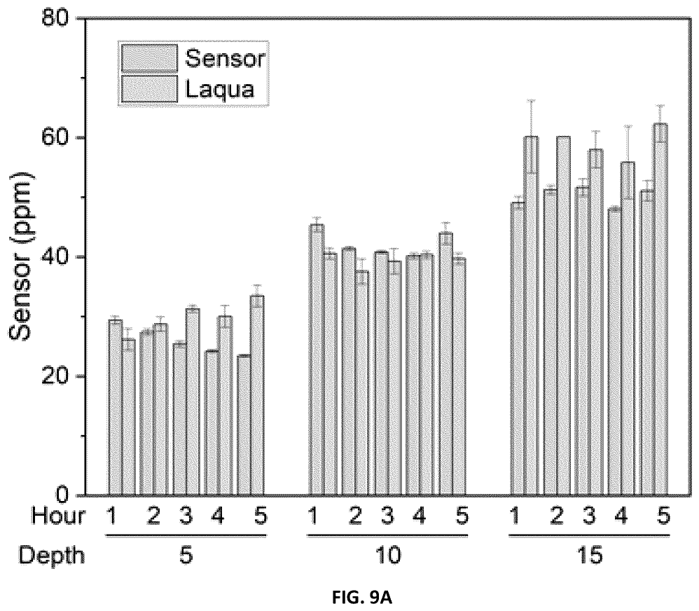

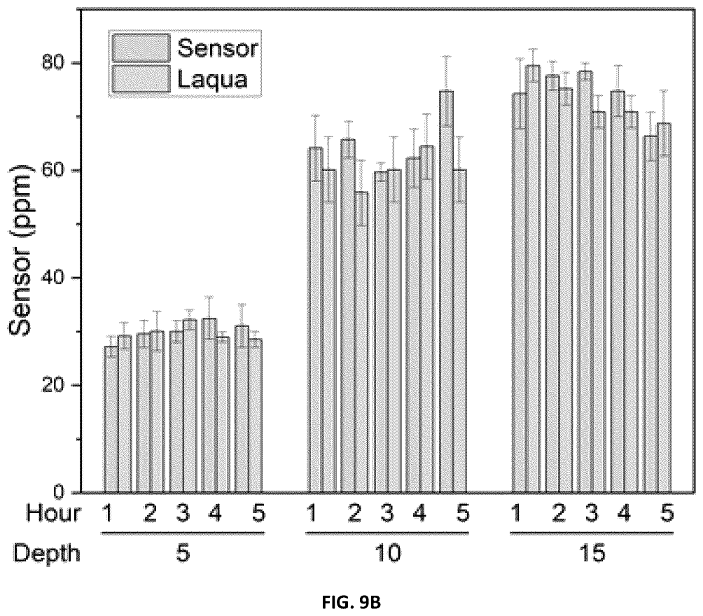

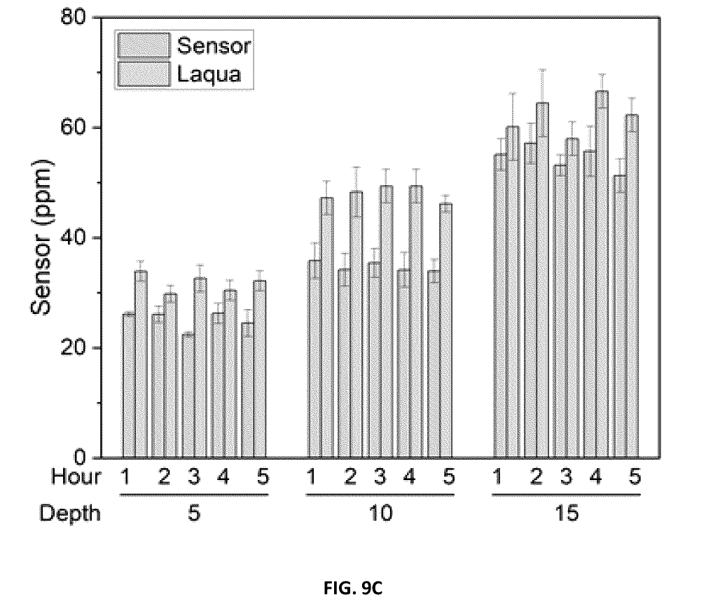

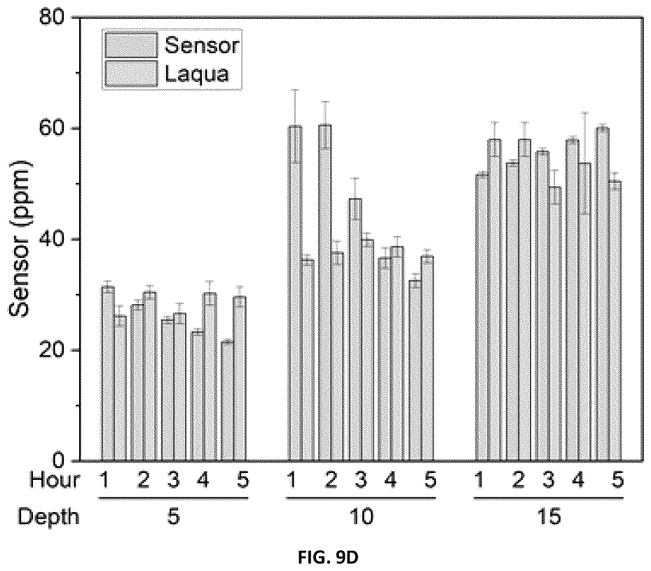

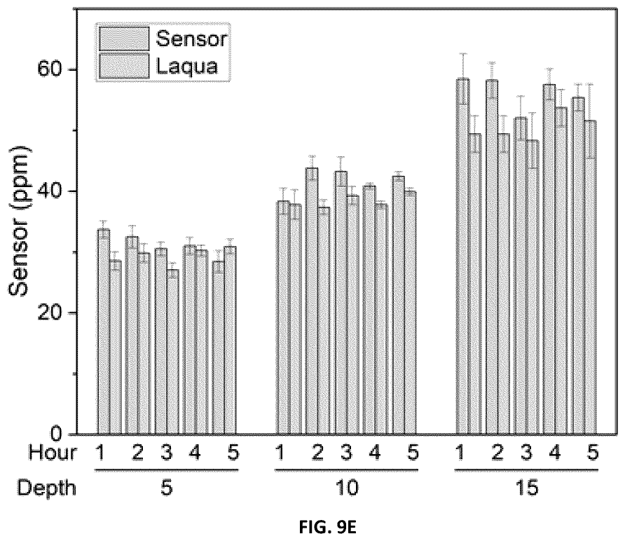

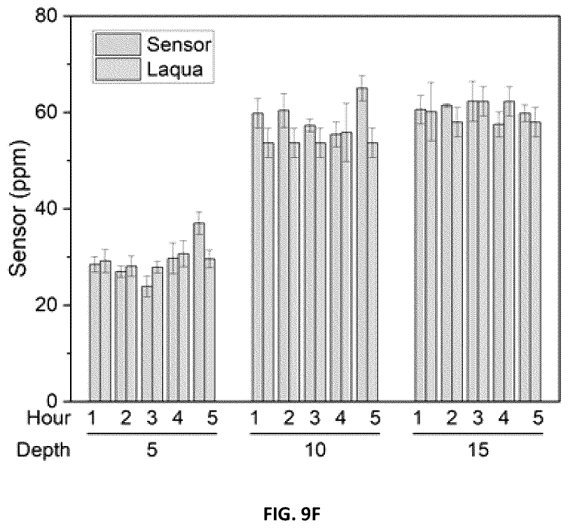

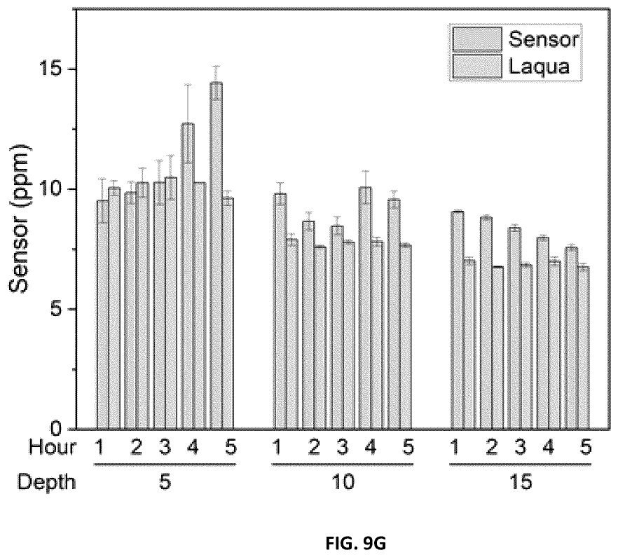

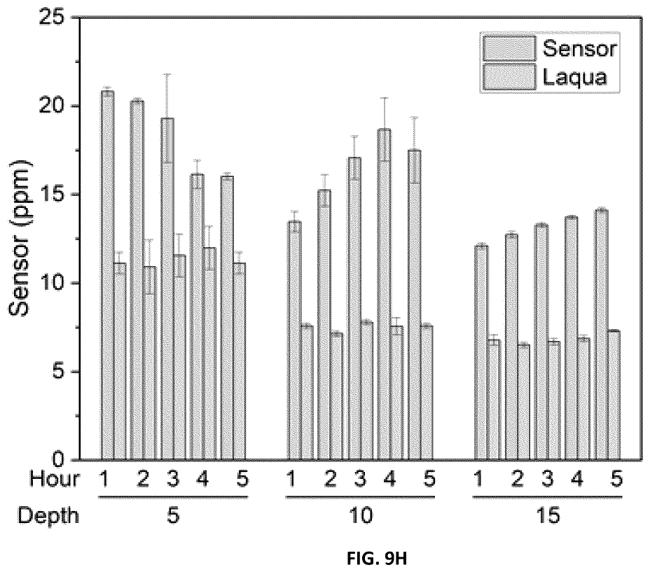

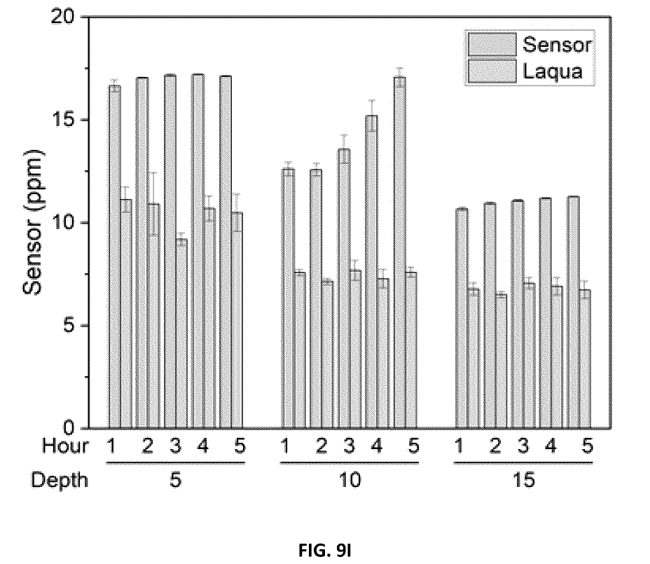

FIG. 9A shows a comparison of nitrate measurements collected using a potentiometric soil sensor array of the disclosure validated against a commercial LaquaTwin sensor at soil depths 5, 10, and 15 cm and over 5 hours in Corn Bare soil.

FIG. 9B shows a comparison of nitrate measurements collected using a potentiometric soil sensor array of the disclosure validated against a commercial LaquaTwin sensor at soil depths 5, 10, and 15 cm and over 5 hours in Corn Plant 1 soil.

FIG. 9C shows a comparison of nitrate measurements collected using a potentiometric soil sensor array of the disclosure validated against a commercial LaquaTwin sensor at soil depths 5, 10, and 15 cm and over 5 hours in Corn Plant 2 soil.

FIG. 9D shows a comparison of nitrate measurements collected using a potentiometric soil sensor array of the disclosure validated against a commercial LaquaTwin sensor at soil depths 5, 10, and 15 cm and over 5 hours in Cowpea Bare soil.

FIG. 9E shows a comparison of nitrate measurements collected using a potentiometric soil sensor array of the disclosure validated against a commercial LaquaTwin sensor at soil depths 5, 10, and 15 cm and over 5 hours in Cowpea Plant 1 soil.

FIG. 9F shows a comparison of nitrate measurements collected using a potentiometric soil sensor array of the disclosure validated against a commercial LaquaTwin sensor at soil depths 5, 10, and 15 cm and over 5 hours in Cowpea Plant 2 soil.

FIG. 9G shows a comparison of nitrate measurements collected using a potentiometric soil sensor array of the disclosure validated against a commercial nitrate sensor at soil depths 5, 10, and 15 cm and over 5 hours in Tomato Bare soil.

FIG. 9H shows a comparison of nitrate measurements collected using a potentiometric soil sensor array of the disclosure validated against a commercial nitrate sensor at soil depths 5, 10, and 15 cm and over 5 hours in Tomato Plant 1 soil.

FIG. 9I shows a comparison of nitrate measurements collected using the potentiometric soil sensor array of the disclosure validated against a commercial nitrate sensor at soil depths 5, 10, and 15 cm and over 5 hours in Tomato Plant 2 soil.

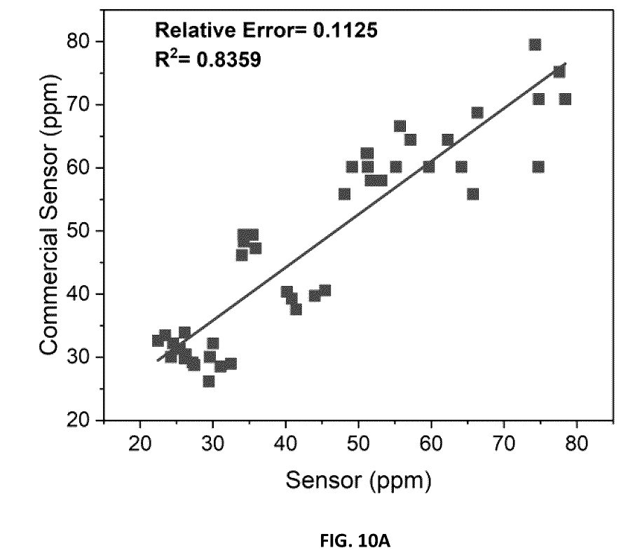

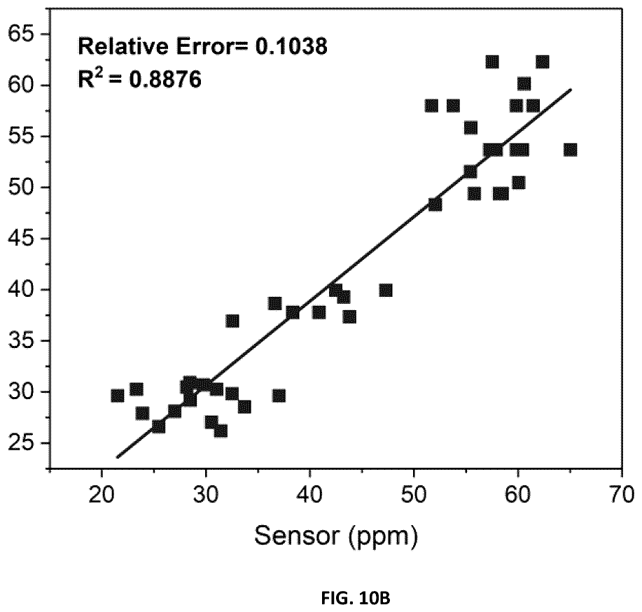

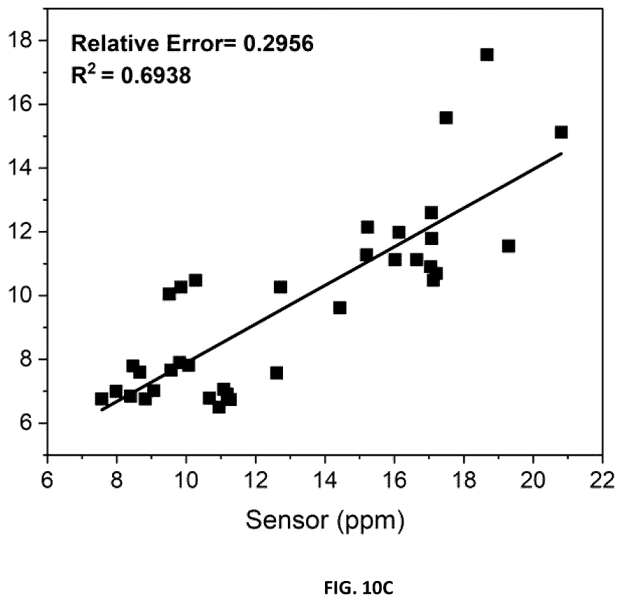

FIG. 10A shows a linear regression plot showing the accuracy of the potentiometric soil sensor array of the disclosure validated against the commercial LaquaTwin sensor in corn plant.

FIG. 10B shows a linear regression plot showing the accuracy of the potentiometric soil sensor array of the disclosure validated against the commercial LaquaTwin sensor in cowpea plant

FIG. 10C shows the accuracy of the potentiometric soil sensor array of the disclosure validated against the commercial LaquaTwin sensor in tomato plant on a linear regression plot.

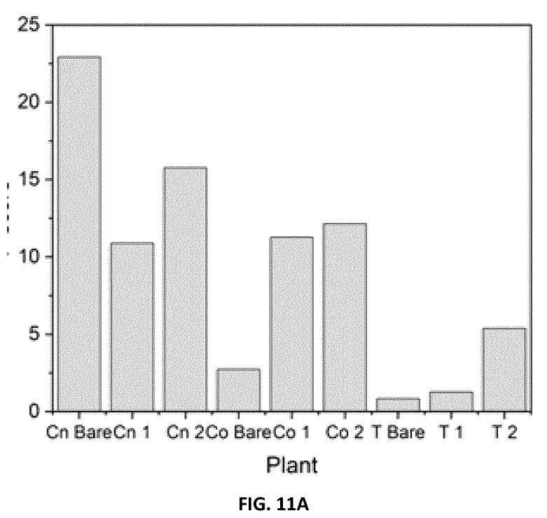

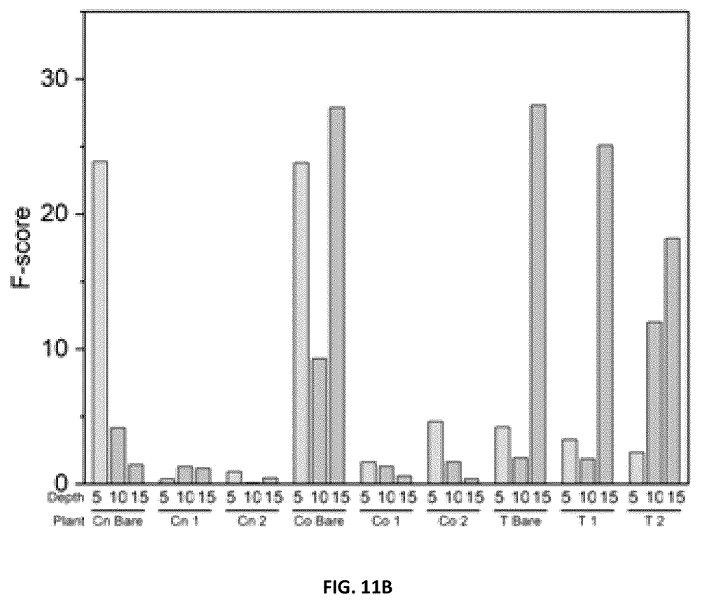

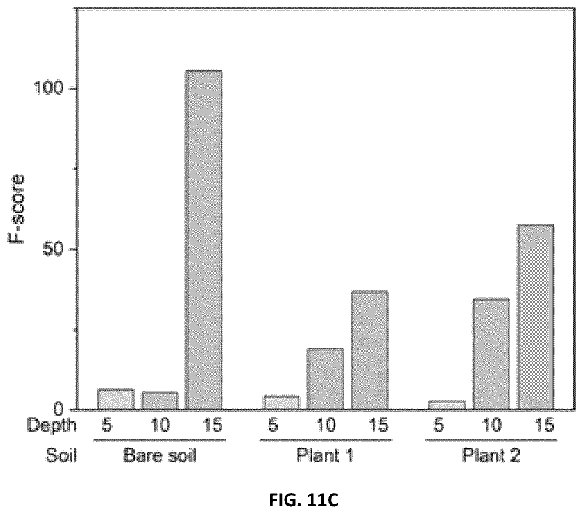

FIG. 11A shows F-scores across soil depths of 5 cm, 10 cm, and 15 cm.

FIG. 11B shows F-scores across moisture levels outlined in Table 1.

FIG. 11C shows F-scores across plant species representing bare soil, plant 1, and plant 2.

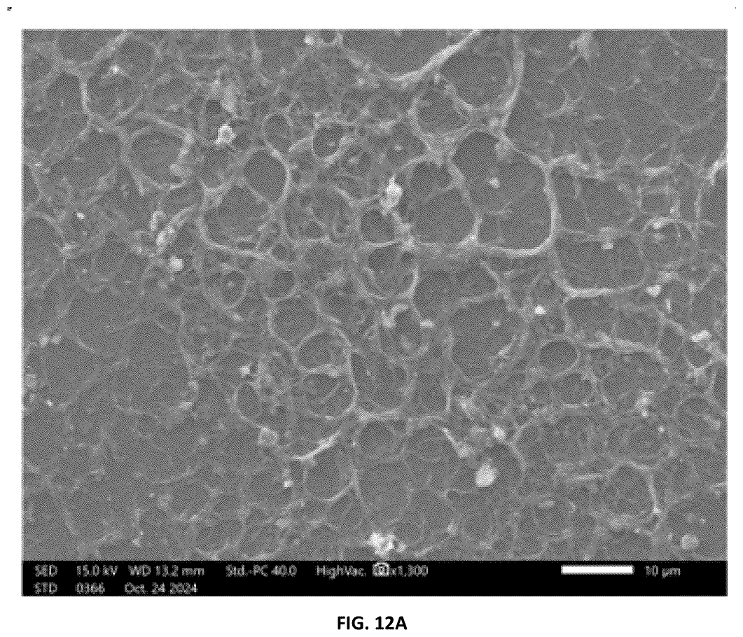

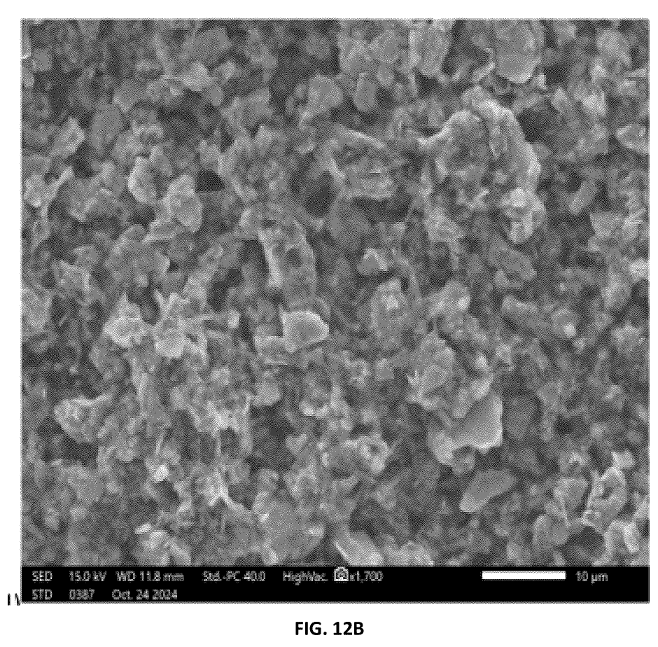

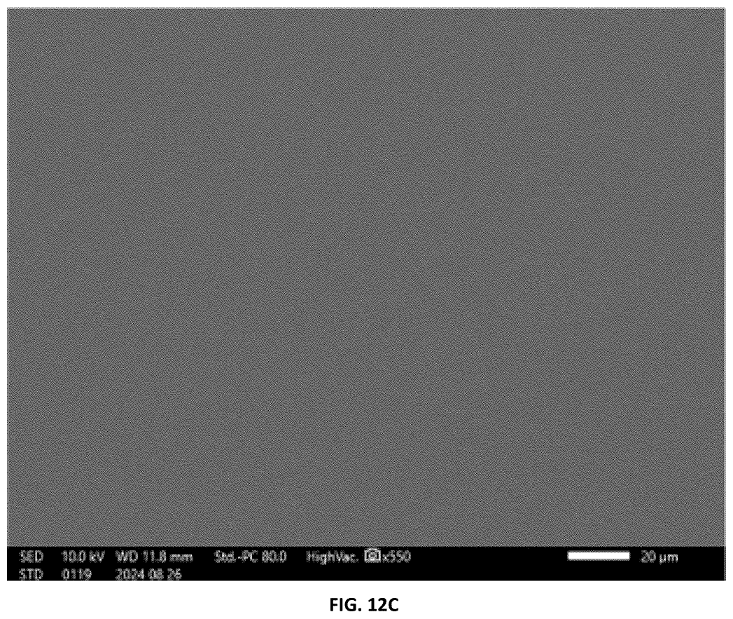

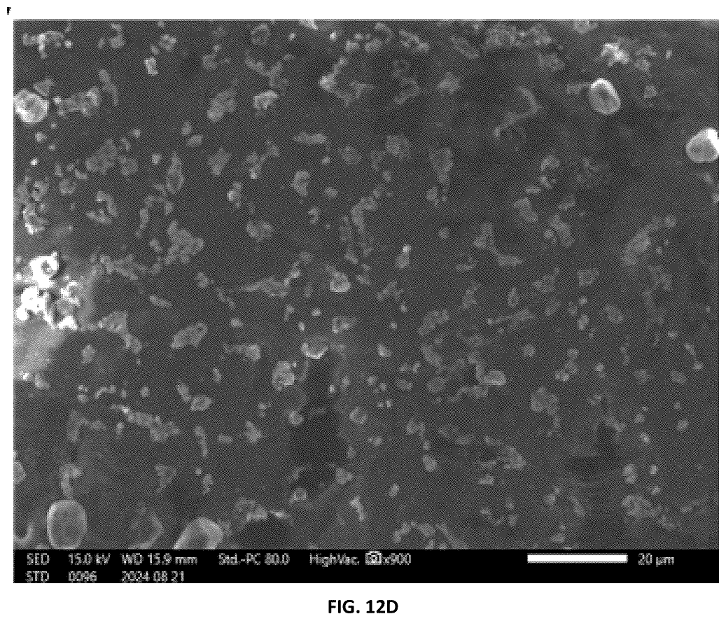

FIG. 12A shows SEM images of SWCNT layer.

FIG. 12B shows SEM images of PoT MoS₂.

FIG. 12C shows SEM images of ISM membrane.

FIG. 12D shows SEM images of the RE membrane.

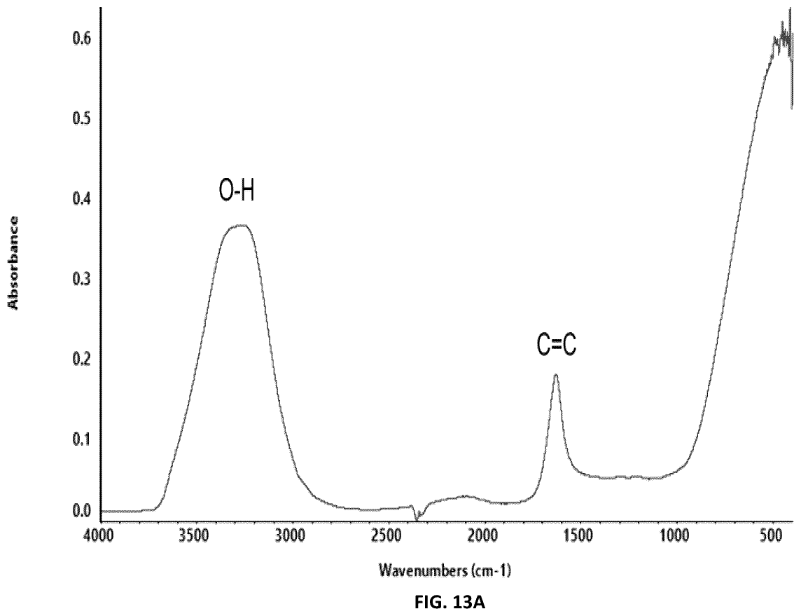

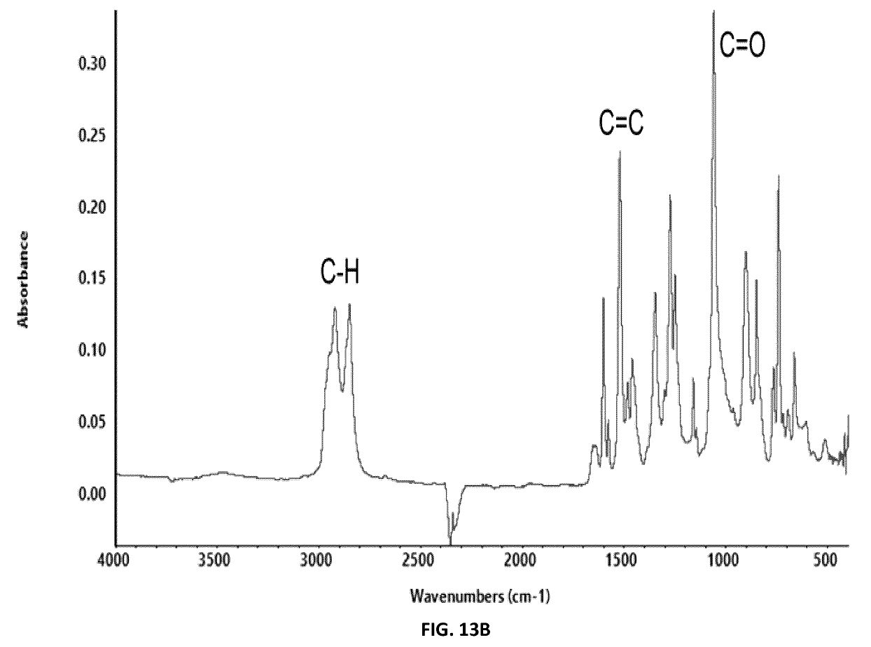

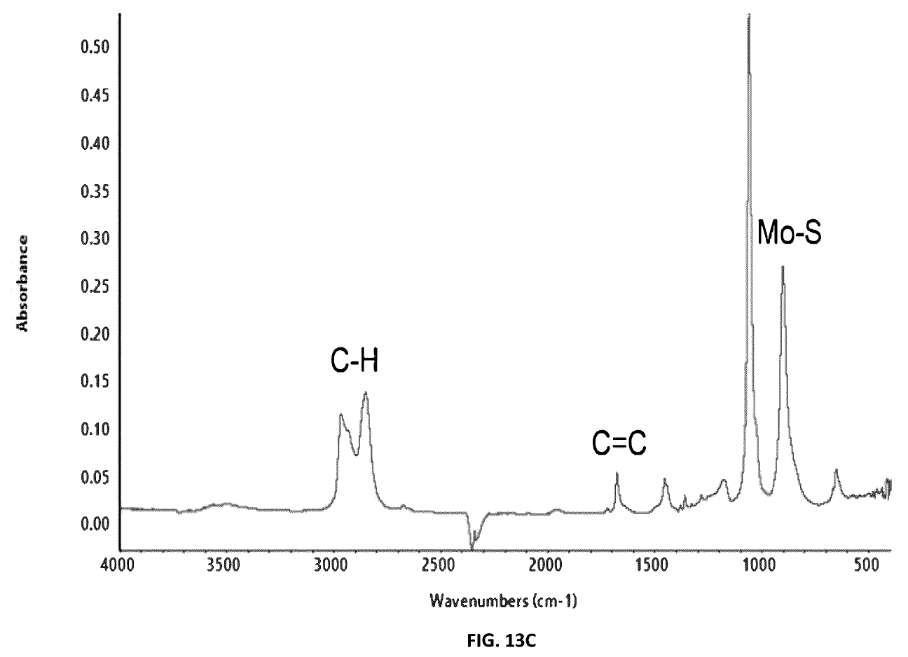

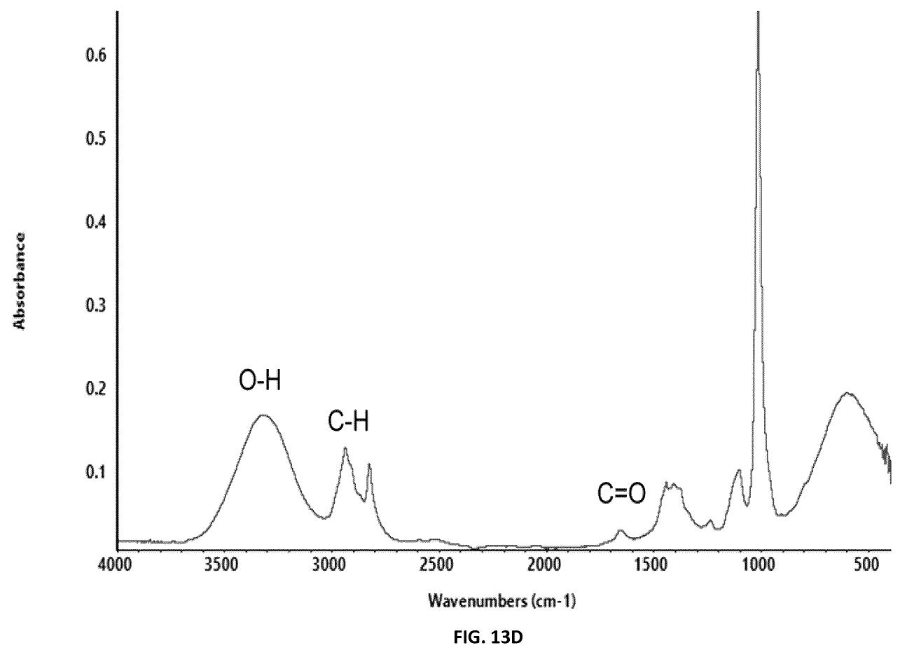

FIG. 13A shows the FTIR spectrum of the SWCNT dispersion.

FIG. 13B shows the FTIR spectrum of the ISM solution.

FIG. 13C shows the FTIR spectrum of the PoT MoS₂ solution.

FIG. 13D shows the FTIR spectrum of the RE membrane containing poly(vinyl butyral) (PVB) and NaCl in methanol.

Note that any one of more of the illustrative components of the real-time monitoring system are optional and the present disclosure includes aspects that contain fewer than all of the illustrated elements.

### DETAILED DESCRIPTION

The disclosure relates to an array of 3D-printed and chemically functionalized potentiometric soil sensors for measuring nitrate levels at different soil depths.

Although the present disclosure is described in detail below, it is to be understood that this disclosure is not limited to the particular methodologies, protocols and reagents described herein as these may vary. It is also to be understood that the terminology used herein is for the purpose of describing particular aspects only and is not intended to limit the scope of the present disclosure.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0′ should be interpreted to include not only the explicitly recited values of about 0.01 to about 2.0, but also include individual values and sub-ranges within the indicated range.

### Definitions

As used herein, the term “soil depth” refers to the depth of soil from the soil surface.

As used herein, the terms “potentiometric sensor,” “potentiometric soil sensor” and “sensor” means a device configured to be inserted into soil to sense one or more conditions, such as nitrate levels. In some embodiments, a plurality of sensors may be configured in a sensor array to detect soil properties at multiple soil depths.

As used herein, the term “real-time monitoring system” a means a device comprising one or more components in addition to the sensor or sensor array, including, but not limited to, connectors, joining parts, a wireless transmitter, and/or a data acquisition system. A real-time monitoring system may include additional components to perform any of the techniques described herein. In some embodiments, the real-time monitoring system is a probe.

As used herein, the terms “approximately” and “about,” as applied to one or more values of interest, refer to a value that is +/−10% of the recited value.

### Real-Time Monitoring System

Different plant types, environmental factors, and soil pH influence plants' preferences for nitrate uptake. Nitrate measurement at different soil depths is also fundamental to optimize fertilization, analyze the root zone, and monitor leaching and crop health. For example, Wang et al. (Field Crops Research, 2015, 183: 117-125), which is fully incorporated herein by reference, teach that yield increases in wheat are negatively correlated with soil-accumulated nitrate at depths of 0-80 and 0-100 cm. Nitrogen intake is affected by various factors, including root distribution and plant species. For example, longer roots can penetrate deeper soil layers and increase the absorption surface area, accessing nitrate that is not available to plants with shorter roots and enhancing nitrate uptake. Additionally, soil moisture levels affect nitrogen uptake by plants. For example, Buljovcic and Engels (Plant and Soil, 2001, 229:125-135), which is incorporated by reference herein, compared nitrate uptake in well-watered and drought conditions and found that a 5% reduction in water content resulted in a 20% decrease in nitrate uptake. The real-time monitoring system of the disclosure provides a wireless, multiplexed, and low-cost system for use in collecting real-time data on nitrogen dynamics in the soil.

In one aspect, the present disclosure includes a potentiometric soil sensor comprising: (i) a substrate; and (ii) a nitrate sensor comprising a nitrate ion-selective membrane (ISM) disposed on the substrate; wherein the nitrate sensor comprises at least one working electrode (WE) and at least one reference electrode (RE); wherein the WE and RE each comprise one or more conductive layers, and wherein the WE and RE are disposed on a surface of the substrate.

In some aspects, the WE and the RE are each disposed in a separate groove on the surface of the substrate. In some aspects, the groove on the front surface are between 1-2 mm in diameter. In some aspects, the two grooves on the front surface are between 0.4-1 mm in depth. In some aspects, the active region is spherical in shape. In some aspects, the active region is between 2.5-3.5 mm in radius. In some aspects, the active region is between 0.4-0.7 mm in depth. In some aspects, the WE and the RE each comprise a connection area and an active region. In some aspects, the WE and RE comprise a carbon coating layer.

In some aspects, the RE further comprises a Ag/AgCl coating layer over the carbon coating layer. In some aspects, the coating layers are a brush coated ink composition. In some aspects, the RE further comprises a single-walled carbon nanotube (SWCNT) coating layer. In some aspects, the WE further comprises a nanocomposite layer over the carbon coating layer. In some aspects, the nanocomposite comprises poly(3-octyl-thiophene) and molybdenum disulfide (PoT-MoS₂). In some aspects, the WE further comprises an ISM coating layer. In some aspects, the WE and RE comprise a passivation layer on at least part of the WE and RE. In some aspects, the passivation layer is disposed on the connection area of the WE and RE.

In some aspects, said substrate comprises a resin material. In some aspects, said substrate comprises a resin material with a post-cured tensile strength of at or greater than 40 MPa, e.g., 41, 45, 50, 55, 60, 65, 70, 75, 80, 85, 90, 95, 100 MPa or any tensile strength above 40. In some aspects, said substrate comprises a resin material with a post-cured flexural strength of at or greater than 60 MPa, e.g., 61, 65, 70, 75, 80, 85, 90, 95, 100 MPa or any post-cured flexural strength above 60. In some aspects, said substrate comprises a resin material with a post-cured notched izod impact strength of at or greater than 35 J/m, e.g., 36, 40, 45, 50, 55, 60, 65, 70, 75, 80, 85, 90, 95, 100. In some aspects, said substrate comprises a resin material with a post-cured heat deflection temperature at 1.8 MPa of between 50-55° C. In some aspects, said substrate comprises a resin material with a post-cured heat deflection temperature at 0.45 MPa of between 60-65° C. In some aspects, said substrate comprises a resin material with a post-cured thermal expansion of between 85-95 μm/m/° C. at 0-150° C.

In one aspect, the present disclosure includes a probe comprising the potentiometric soil sensor of the disclosure. In some aspects, the probe comprises an array of the potentiometric soil sensors. In some aspects, the array comprises at least two of the potentiometric sensors. In some aspects, the array comprises at least three of the potentiometric sensors. In some aspects, each potentiometric soil sensor is vertically stacked. In some aspects, each potentiometric soil sensor is mechanically attached to at least one connector. In some aspects, each connector is positioned on top of each potentiometric soil sensor and wherein the active region of the WE and the active region of the RE are positioned away from the connector positioned on top of the potentiometric sensor.

In some aspects, the probe comprises a plurality of potentiometric soil sensors and connectors, wherein the potentiometric soil sensors and the connectors are assembled in an alternating pattern. In some aspects, the probe comprises joining parts, said joining parts structurally reinforcing the mechanical attachment of each connector to each potentiometric sensor. In some aspects, the probe provides multi-depth nitrate concentration measurements. In some aspects, each potentiometric soil sensor provides a different soil depth nitrate concentration measurement.

In one aspect, the present disclosure includes a real-time monitoring system comprising the following structural assembly in a top to bottom direction: [C1]-[S1]-[C2]-[S2]-[C3]-[S3], wherein C1 is a first connector, S1 is a first potentiometric sensor, C2 is a second connector, S2 is a second potentiometric sensor, C3 is a third connector, S3 is a third potentiometric sensor; and wherein each connector is mechanically attached to each potentiometric soil sensor by a joining part.

In some aspects, S3 includes a cone tip configured to penetrate a soil. In some aspects, the potentiometric soil sensor is 1-4 cm in vertical length. In some aspects, the potentiometric soil sensor is about 2 cm in vertical length. In some aspects, C1 is about 3 cm in vertical length, C2 is about 2 cm in vertical length, and C3 is about 2 cm in vertical length. In some aspects, the real-time monitoring system detects soil nitrate concentration at 5 cm, 10 cm, and 15 cm soil depth.

In some aspects, each potentiometric probe further comprises a data acquisition circuit. In some aspects, the data acquisition circuit comprises an analog front end (AFE), a microcontroller unit (MCU), and a data transmission circuit. In some aspects, the AFE further comprises at least on instrumentation amplified (IN-AMP), at least one reference voltage and at least one lowpass filter (LPF). In some aspects, the AFE receives the soil nitrate concentration measurement from the RE and WE.

In some aspects, the probe further comprises a wireless transmitter for transmitting the soil nitrate concentration measurement signal to a remote location. In some aspects, the wireless transmitter is an integrated dual-band transceiver antenna system. In some aspects, the wireless transmitter comprises two frequency bands. In some aspects, the data acquisition circuit comprises a non-transitory computer readable medium communicatively coupled to the processor, the non-transitory computer readable medium having stored thereon computer software comprising a set of instructions that, when executed by the processor, causes an electrode control unit to: (i) receive electrode data from each of the AFE; and (2) send, via the wireless transmitter, the potentiometric soil sensor data to an external device.

In some aspects, the potentiometric soil sensor data are sent, via the wireless transmitter, to an Internet of Things (IoT) cloud server configured to interact with one or more IoT-capable devices.

### Method for Making a Real-Time Monitoring System

In one aspect, the present disclosure includes a method for making at least one potentiometric soil sensor comprising the steps of:
a. 3D printing a substrate including at least two grooves on a front surface transversing from one end of the substrate to the other end of the substrate along a vertical direction; and
b. disposing a WE comprising an active region and a connection region and a RE comprising an active region and a connection region into the two grooves on the front surface of the substrate.

In some aspects, the WE and the RE are brush coated with carbon (C) ink. In some aspects, the C ink-coated RE is brush coated with Ag/AgCl ink. In some aspects, the active region of the C ink-coated WE is drop casted with a nanocomposite. In some aspects, the nanocomposite-coated active region of WE is drop casted with nitrate ion-selective membrane ISM. In some aspects, the Ag/AgCl-coated RE is spray deposited with single-walled carbon nanotubes (SWCNT). In some aspects, the SWCNT coated RE is drop casted with a RE membrane. In some aspects, the connection region of the WE and the RE is coated with a passivation layer.

In one aspect, the present disclosure includes a method for making at least one potentiometric soil sensor comprising the steps of:
a. 3D printing a substrate including at least two grooves on a front surface transversing from one end of the substrate to the other end of the substrate along a vertical direction;
b. disposing a WE comprising an active region and a connection region and a RE comprising an active region and a connection region into the two grooves on the front surface of the substrate;
c. brush coating the WE and the RE with carbon (C) ink;
d. brush coating Ag/AgCl ink on the C ink-coated RE of (c);
e. drop casting a nanocomposite on the active region of the C ink-coated WE of (c);
f. drop casting ISM on the nanocomposite-coated active region of WE of (e)
g. spray depositing single-walled carbon nanotubes (SWCNT) on top of the Ag/AgCl-coated RE of (d);
h. drop casting of RE membrane on top of the SWCNT coated RE of (g); and
i. forming a passivation layer on the connection region of the WE and the RE.

In some aspects, the nanocomposite is drop-casted on the WE using a robotic spray dispenser. In some aspects, the nanocomposite comprises a alkylthiophene and a transition metal dichalcogenide. In some aspects, the alkylthiophene is poly(3-octyl-thiophene) (PoT). In some aspects, the transition metal dichalcogenide is molybdenum disulfide (MoS₂).

In some aspects, said nanocomposite is prepared by mixing PoT with MoS₂ in a polar aprotic solvent to make a mixture. In some aspects, said polar aprotic solvent is tetrahydrofuran (THF). In some aspects, the mixture is further (1) magnetically stirred the mixture at 600-1000 rpm for at least 20 minutes to make a solution; and (2) horn sonicated at 30-50% intensity for at least 20 minutes.

In some aspects, the ISM comprises a organophosphorus compound, a cellulose compound, an organic solvent, a high-strength thermoplastic material, an ionic surfactant and a polar aprotic solvent. In some aspects, the ISM is prepared by mixing the organophosphorus compound, the cellulose compound, the an organic solvent, the high-strength thermoplastic material, and the ionic surfactant in the polar aprotic solvent. In some aspects, the organophosphorus compound is methyltriphenylphosphonium bromide. In some aspects, the cellulose compound is nitrocellulose. In some aspects, the organic solvent is 2-nitrophenyl octyl ether. In some aspects, the high-strength thermoplastic material is polyvinyl chloride (PVC). In some aspects, the ionic surfactant is tridodecylmethylammonium nitrate. In some aspects, the polar aprotic solvent is tetrahydrofuran (THF).

In some aspects, the SWCNT are spray-coated on the RE using a robotic dispenser over the electrode surface 50-150 times at a bed temperature of 50-90° C. In some aspects, said SWCNT are prepared by (1) making a solvent by mixing SDS with deionized (DI) water, (2) stirring the solvent of (1), (3) adding SWCNT to the solvent of (2) to make a solution, (4) mixing the solution of (3). In some aspects, said SWCNT are prepared by (1) making a solvent by mixing 250 mg of 0.5% SDS with deionized (DI) water, (2) stirring the solvent of (1) for 1 hour at 600 rpm, (3) adding 25 mg of 0.05% SWCNT to the solvent of (2) to make a solution, (4) stirring the solution of (3) for 30 minutes, (5) horn-sonicating the solution of (4) an ice-filled beaker for 30 minutes at 50% intensity, and (6) centrifuging the solution of (5) at 13,200 rpm for 110 minutes.

In some aspects, the RE is first coated with the SWCNT and then coated with a RE membrane. In some aspects, the RE membrane comprises a resin, NaCl and methanol. In some aspects, the resin is poly(vinyl butyral) (PVB). In some aspects, the RE membrane is prepared by (1) preparing a solution by dissolving 0.5-2 g of poly(vinyl butyral) (PVB) and 0.5-2 g of NaCl in 10-25 mL of methanol, and (2) sonicating in an ice bath for 20-40 minutes. In some aspects, the RE membrane coated RE is resistant to chlorine leaching.

In some aspects, the substrate is 0.5-10 cm in vertical length. In some aspects, the substrate is 1-4 cm in vertical length. In some aspects, the substrate includes a cone tip configured to penetrate a soil. In some aspects, the substrate is rectangular in shape. In some aspects, the substrate comprises a resin material. In some aspects, the resin material has a post-cured tensile strength of greater than 40 MPa. In some aspects, the resin material has a post-cured flexural strength of greater than 60 MPa. In some aspects, the resin material has a post-cured notched izod impact strength of greater than 35 J/m. In some aspects, the resin material has a post-cured heat deflection temperature at 1.8 MPa of between 50-55° C. In some aspects, the resin material has a post-cured heat deflection temperature at 0.45 MPa of between 60-65° C. In some aspects, the resin material has a post-cured thermal expansion of between 85-95 μm/m/° C. at 0-150° C.

In some aspects, the two grooves on the front surface are between 0.5-10 mm in diameter. In some aspects, the two grooves on the front surface are between 1-2 mm in diameter. In some aspects, the two grooves on the front surface are between 0.4-1 mm in depth.

In some aspects, the active region is spherical in shape. In some aspects, the active region is between 1-10 mm in radius. In some aspects, the active region is between 0.1 to 2 mm in depth. In some aspects, the active region is spherical in shape. In some aspects, the active region is between 2-5 mm in radius. In some aspects, the active region is between 0.2-1 mm in depth. In some aspects, the active region is spherical in shape. In some aspects, the active region is between 2.5-3.5 mm in radius. In some aspects, the active region is between 0.4-0.7 mm in depth.

In one aspect, the present disclosure includes a method for making a real-time monitoring system for in situ soil nitrate concentration analysis comprising:
a) making at least one potentiometric soil sensor comprising the steps of:
   i) 3D printing a substrate including at least two grooves on a front surface and two grooves in the back surface transversing from one end of the substrate to the other end of the substrate along a vertical direction;
   ii) inserting a WE comprising an active region and a connection region and a RE comprising an active region and a connection region into the grooves of the substrate;
   iii) brush coating the WE and the RE with C ink;
   iv) brush coating Ag/AgCl ink on the C ink-coated RE of step (iii);
   v) drop casting a nanocomposite on the active region of the C ink-coated WE of step (iii);
   vi) drop casting ISM on the nanocomposite-coated active region of WE of step (v);
   vii) spray depositing SWCNTs on top of the Ag/AgCl-coated RE of step (iv);
   viii) drop casting of RE membrane on top of the SWCNT coated RE of step (vii); and
   ix) forming a passivation layer on the connection region of the WE and the RE;
b) making at least one connector; and
c) mechanically attaching the least one potentiometric soil sensor to the at least one connector.

In some aspects, the nanocomposite is drop-casted on the WE using a robotic spray dispenser. In some aspects, the nanocomposite comprises an alkylthiophene and a transition metal dichalcogenide. In some aspects, the nanocomposite comprises poly(3-octyl-thiophene) and molybdenum disulfide (PoT-MoS₂). In some aspects, said nanocomposite is prepared by mixing the alkylthiophene and transition metal dichalcogenide. In some aspects, said nanocomposite is prepared by mixing PoT with MoS₂ in a polar aprotic solvent to make a mixture. In some aspects, said polar aprotic solvent is tetrahydrofuran (THF). In some aspects, the mixture is further (1) stirred to make a solution; and (2) sonicated. In some aspects, the mixture is further (1) magnetically stirred at 500 to 2000 rpm for 10 to 60 minutes to make a solution; and (2) horn sonicated at 30-50% intensity for 10 to 60 minutes. In some aspects, the mixture is further (1) magnetically stirred at 800 rpm for 30 minutes to make a solution; and (2) horn sonicated at 40% intensity for 30 minutes.

In some aspects, the ISM comprises an organophosphorus compound, a cellulose compound, an organic solvent, a high-strength thermoplastic material, an ionic surfactant and a polar aprotic solvent. In some aspects, the organophosphorus compound is methyltriphenylphosphonium bromide, the cellulose compound is nitrocellulose, the organic solvent is 2-nitrophenyl octyl ether, the high-strength thermoplastic material is polyvinyl chloride, the ionic surfactant is tridodecylmethylammonium nitrate and the polar aprotic solvent is THF.

In some aspects, the ISM is prepared by mixing an organophosphorus compound, a cellulose compound, an organic solvent, a high-strength thermoplastic material, an ionic surfactant and a polar aprotic solvent. In some aspects, the ISM is prepared by mixing methyltriphenylphosphonium bromide, nitrocellulose, 2-nitrophenyl octyl ether, polyvinyl chloride, and tridodecylmethylammonium nitrate in a solvent. In some aspects, the ISM is prepared by mixing 5 to 20 mg methyltriphenylphosphonium bromide, 60 to 120 mg nitrocellulose, 0.5 to 5 ml 2-nitrophenyl octyl ether, 300 to 600 mg polyvinyl chloride, and 50 to 200 mg tridodecylmethylammonium nitrate in THF. In some aspects, the ISM is prepared by mixing 12.5 mg methyltriphenylphosphonium bromide, 96.5 mg nitrocellulose, 1.73 ml 2-nitrophenyl octyl ether, 470 mg polyvinyl chloride, and 125 mg tridodecylmethylammonium nitrate in THF.

In some aspects, the SWCNT are spray-coated on the RE using a robotic dispenser over the electrode surface at least 80 times at a bed temperature of 50-90° C. In some aspects, said SWCNT are prepared by (1) making a solvent by mixing SDS with deionized (DI) water, (2) stirring the solvent of (1) for at least 30 minutes at 400-800 rpm, (3) adding 10-40 mg of 0.03-0.1% SWCNT to the solvent of (2) to make a solution, (4) mixing the solution of (3). In some aspects, said SWCNT are prepared by (1) making a solvent by mixing 200-300 mg of 0.03-0.1% SDS with deionized (DI) water, (2) stirring the solvent of (1) for 45-75 min at 400-800 rpm, (3) adding 20-30 mg of 0.05% SWCNT to the solvent of (2) to make a solution, (4) stirring the solution of (3) for 20-40 minutes, (5) horn-sonicating the solution of (4) an ice-filled beaker for 20-40 minutes at 40-60% intensity, and (6) centrifuging the solution of (5) at 12,000-14,000 rpm for 100-120 minutes.

In some aspects, the RE is first coated with the SWCNT and then coated with a RE membrane. In some aspects, the RE comprising resin, NaCl and methanol. In some aspects, the RE membrane is prepared by (1) preparing a solution by dissolving 1.58 g of poly(vinyl butyral) (PVB) and 1 g of NaCl in 20 mL of methanol, and (2) sonicating in an ice bath for 30 minutes.

In some aspects, the real-time monitoring system comprises the following structural assembly in a top to bottom direction: [C1]-[S1]-[C2]-[S2]-[C3]-[S3], wherein C1 is a first connector, S1 is a first potentiometric sensor, C2 is a second connector, S2 is a second potentiometric sensor, C3 is a third connector, S3 is a third potentiometric sensor. In some aspects, the real-time monitoring system further comprises at least one joining part, said joining part structurally reinforcing the mechanical attachment of each connector to each potentiometric sensor. In some aspects, the C1 is 3 cm in vertical length, C2 is 2 cm in vertical length, and C3 is 2 cm in vertical length. In some aspects, the the real-time monitoring system provides multi-depth nitrate concentration measurements. In some aspects, the each potentiometric soil sensor provides a different soil depth nitrate concentration measurement. In some aspects, the real-time monitoring system detects soil nitrate concentration at 5 cm, 10 cm, and 15 cm soil depth.

In some aspects, the substrate comprises a resin material. In some aspects, the resin material has a post-cured tensile strength of 40-50 MPa. In some aspects, the resin material has a post-cured flexural strength of 60-70 MPa. In some aspects, the resin material has a post-cured notched izod impact strength of greater than 35-45 J/m. In some aspects, the resin material has a post-cured heat deflection temperature at 1.8 MPa of between 50-55° C. In some aspects, the resin material has a post-cured heat deflection temperature at 0.45 MPa of between 60-65° C. In some aspects, the resin material has a post-cured thermal expansion of between 85-95 μm/m/° C. at 0-150° C.

In some aspects, the two grooves on the front surface are between 1-2 mm in diameter. In some aspects, the two grooves on the front surface are between 0.4-1 mm in depth. In some aspects, the active region is spherical in shape. In some aspects, the active region is between 2.5-3.5 mm in radius. In some aspects, the active region is between 0.4-0.7 mm in depth. In some aspects, the potentiometric soil sensor is 1-4 cm in vertical length. In some aspects, the potentiometric soil sensor is 2 cm in vertical length.

In some aspects, the real-time monitoring system further comprises a data acquisition circuit. In some aspects, the data acquisition circuit comprises an analog front end (AFE), a microcontroller unit (MCU), and a data transmission circuit. In some aspects, the AFE receives the soil nitrate concentration measurement from the RE and WE.

In some aspects, the real-time monitoring system further comprises a wireless transmitter for transmitting the soil nitrate concentration measurement signal to a remote location. In some aspects, the wireless transmitter is an integrated dual-band transceiver antenna system. In some aspects, the wireless transmitter comprises two frequency bands.

### Method for Selectively Measuring Nitrate in Soil

In one aspect, the present disclosure includes a method of selectively measuring nitrate in soil, comprising: inserting a probe comprising at least one potentiometric soil sensor into a soil; generating an electrical potential between a reference electrode (RE) and a working electrode (WE), wherein the potential is based on nitrate levels in the soil; producing an electrical signal in response to the electrical potential between the RE and the WE, wherein said electrical signal indicates the soil nitrate concentration; transmitting the electrical signal to a data acquisition circuit to apply a calibration operation; determining the soil nitrate concentration based on the calibration operation.

In some aspects, the probe contains a plurality of potentiometric sensors. In some aspects, the probe further comprises at least one wireless transmitter capable of transmitting the soil nitrate concentration measurement signal to a remote location. In some aspects, the wireless transmitter is configured to transmit soil measurement data from each of the plurality of potentiometric sensors.

In some aspects, the data acquisition circuit comprises an analog front end (AFE), a microcontroller unit (MCU), and a data transmission circuit. In some aspects, the data acquisition circuit applies at least one reference voltage during the calibration operation, the reference voltage providing a reference against which the potential measured by the RE and WE is compared. In some aspects, the electrical potential generated by the RE and WE ranges from −100 mV to +500 mV in response to the soil nitrate concentration.

In some aspects, the probe continuously monitors the soil nitrate concentrations. In some aspects, the probe continuously monitors the soil nitrate concentrations at a frequency recorded between once a second to once a minute. In some aspects, the probe continuously monitors the soil nitrate concentrations at a frequency recorded between once a second to once a day. In some aspects, the probe continuously monitors the soil nitrate concentrations at a frequency recorded between once a second to once an hour. In some aspects, the probe provides multi-depth nitrate concentration measurements.

The present disclosure includes the following non-limiting numbered items:

1. A potentiometric soil sensor comprising:
(i) a substrate; and
(ii) a nitrate sensor comprising a nitrate ion-selective membrane (ISM) disposed on the substrate;
   wherein the nitrate sensor comprises at least one working electrode (WE) and at least one reference electrode (RE);
   wherein the WE and RE each comprise one or more conductive layers, and
   wherein the WE and RE are disposed on a surface of the substrate.
   2. The potentiometric soil sensor of item 1, wherein the WE and the RE are each disposed in a separate groove on the surface of the substrate.
   3. The potentiometric soil sensor of item 1 or item 2, wherein the WE and the RE each comprise a connection area and an active region.
   4. The potentiometric soil sensor of any one or combination of items herein, wherein the WE and RE comprise a carbon coating layer.
   5. The potentiometric soil sensor of item 4, wherein the RE further comprises a silver or silver chloride (Ag/AgCl) coating layer over the carbon coating layer.
   6. The potentiometric soil sensor of item 4 or item 5, wherein the coating layers are a brush coated ink composition.
   7. The potentiometric soil sensor of item 5, wherein the RE further comprises a single-walled carbon nanotube (SWCNT) coating layer.
   8. The potentiometric soil sensor of any one or combination of items herein, wherein the WE further comprises a nanocomposite layer over the carbon coating layer.
   9. The potentiometric soil sensor of item 8, wherein the nanocomposite comprises poly(3-octyl-thiophene) and molybdenum disulfide (PoT-MoS2).
   10. The potentiometric soil sensor of any preceding item, wherein the WE and RE comprise a passivation layer on at least part of the WE and RE.
   11. The potentiometric soil sensor of item 10, wherein the passivation layer is disposed on the connection area of the WE and RE.
   12. The potentiometric soil sensor of any one or combination of items herein, wherein said substrate comprises a resin material.
   13. The potentiometric soil sensor of any one or combination of items herein, wherein said substrate comprises a resin material with a post-cured tensile strength of greater than 40 MPa.
   14. The potentiometric soil sensor of any one or combination of items herein, wherein said substrate comprises a resin material with a post-cured flexural strength of greater than 60 MPa.
   15. The potentiometric soil sensor of any one or combination of items herein, wherein said substrate comprises a resin material with a post-cured notched izod impact strength of greater than 35 J/m.
   16. The potentiometric soil sensor of any one or combination of items herein, wherein said substrate comprises a resin material with a post-cured heat deflection temperature at 1.8 MPa of between 50-55° C.
   17. The potentiometric soil sensor of any one or combination of items herein, wherein said substrate comprises a resin material with a post-cured heat deflection temperature at 0.45 MPa of between 60-65° C.
   18. The potentiometric soil sensor of any one or combination of items herein, wherein said substrate comprises a resin material with a post-cured thermal expansion of between 85-95 m/m/° C. at 0-150° C.
   19. The potentiometric soil sensor of any one or combination of items herein, wherein said groove on the front surface are between 1-2 mm in diameter.
   20. The potentiometric soil sensor of any one or combination of items herein, wherein said two grooves on the front surface are between 0.4-1 mm in depth.
   21. The potentiometric soil sensor of any one or combination of items herein, wherein said active region is spherical in shape.
   22. The potentiometric soil sensor of any one or combination of items herein, wherein said active region is between 2.5-3.5 mm in radius.
   23. The potentiometric soil sensor of any one or combination of items herein, wherein said active region is between 0.4-0.7 mm in depth.
   24. A probe comprising the potentiometric soil sensor of any one or combination of items herein.
   25. The probe of item 24, comprising an array of the potentiometric soil sensors.
   26. The probe of item 25, wherein the array comprises at least two of the potentiometric sensors.
   27. The probe of item 26, wherein the array comprises at least three of the potentiometric sensors.
   28. The probe of any one or combination of items herein, wherein each potentiometric soil sensor is vertically stacked.
   29. The probe of any one or combination of items herein, wherein each potentiometric soil sensor is mechanically attached to at least one connector.
   30. The probe of item 29, wherein each connector is positioned on top of each potentiometric soil sensor and wherein the active region of the WE and the active region of the RE are positioned away from the connector positioned on top of the potentiometric sensor.
   31. The probe of any one or combination of items herein, comprising a plurality of potentiometric soil sensors and connectors, wherein the potentiometric soil sensors and the connectors are assembled in an alternating pattern.
   32. The probe of any one or combination of items herein, further comprising joining parts, said joining parts structurally reinforcing the mechanical attachment of each connector to each potentiometric sensor.
   33. The probe of any one or combination of items herein, wherein the probe provides multi-depth nitrate concentration measurements.
   34. The probe of any one or combination of items herein, wherein each potentiometric soil sensor provides a different soil depth nitrate concentration measurement.
   35. The probe of any one or combination of items herein, wherein the real-time monitoring system comprises the following structural assembly in a top to bottom direction: [C1]-[S1]-[C2]-[S2]-[C3]-[S3], wherein C1 is a first connector, S1 is a first potentiometric sensor, C2 is a second connector, S2 is a second potentiometric sensor, C3 is a third connector, S3 is a third potentiometric sensor; and wherein each connector is mechanically attached to each potentiometric soil sensor by a joining part.
   36. The probe of item 35, wherein S3 includes a cone tip configured to penetrate a soil.
   37. The probe of any one or combination of items herein, wherein the potentiometric soil sensor is 1-4 cm in vertical length.
   38. The probe of any one or combination of items herein, wherein the potentiometric soil sensor is 2 cm in vertical length.
   39. The probe of any one or combination of items herein, wherein C1 is 3 cm in vertical length, C2 is 2 cm in vertical length, and C3 is 2 cm in vertical length.
   40. The probe of any one or combination of items herein, wherein the real-time monitoring system detects soil nitrate concentration at 5 cm, 10 cm, and 15 cm soil depth.
   41. The probe of any one or combination of items herein, further comprising a data acquisition circuit.
   42. The probe of any one or combination of items herein, wherein the data acquisition circuit comprises an analog front end (AFE), a microcontroller unit (MCU), and a data transmission circuit.
   43. The probe of item 42, wherein the AFE further comprises at least one instrumentation amplified (IN-AMP), at least one reference voltage and at least one lowpass filter (LPF).
   44. The probe of item 43, wherein the AFE recieves the soil nitrate concentration measurement from the RE and WE.
   45. The probe of any one or combination of items herein, further comprising a wireless transmitter for transmitting the soil nitrate concentration measurement signal to a remote location.
   46. The probe of item 45, wherein the wireless transmitter is an integrated dual-band transceiver antenna system.
   47. The probe of any one or combination of items herein, wherein the wireless transmitter comprises two frequency bands.
   48. The probe of any one or combination of items herein, wherein the data acquisition circuit comprises a non-transitory computer readable medium communicatively coupled to a processor, the non-transitory computer readable medium having stored thereon computer software comprising a set of instructions that, when executed by the processor, causes an electrode control unit to:
receive electrode data from each of the AFE; and
send, via the wireless transmitter, the potentiometric soil sensor data to an external device.
   49. The probe of any one or combination of items herein, wherein the potentiometric soil sensor data are sent, via the wireless transmitter, to an Internet of Things (IoT) cloud server configured to interact with one or more IoT-capable devices.
   50. A method for making at least one potentiometric soil sensor comprising the steps of:
a) 3D printing a substrate including at least two grooves on a front surface transversing from one end of the substrate to the other end of the substrate along a vertical direction;
b) disposing a WE comprising an active region and a connection region and a RE comprising an active region and a connection region into the two grooves on the front surface of the substrate.
   51. The method of item 50, wherein the WE and the RE are brush coated with carbon (C) ink.
   52. The method of item 51, wherein the C ink-coated RE is brush coated with Ag/AgCl ink.
   53. The method of any one or combination of items herein, wherein the active region of the C ink-coated WE is drop casted with a nanocomposite.
   54. The method of item 53, wherein the nanocomposite-coated active region of WE is drop casted with nitrate ion-selective membrane ISM.
   55. The method of any one or combination of items herein, wherein the Ag/AgCl-coated RE is spray deposited with single-walled carbon nanotubes (SWCNT).
   56. The method of item 55, wherein the SWCNT coated RE is drop casted with a RE membrane.
   57. The method of any one or combination of items herein, wherein the connection region of the WE and the RE is coated with a passivation layer.
   58. A method for making at least one potentiometric soil sensor comprising the steps of:
a) 3D printing a substrate including at least two grooves on a front surface transversing from one end of the substrate to the other end of the substrate along a vertical direction;
b) disposing a WE comprising an active region and a connection region and a RE comprising an active region and a connection region into the two grooves on the front surface of the substrate;
c) brush coating the WE and the RE with carbon (C) ink;
d) brush coating Ag/AgCl ink on the C ink-coated RE of step (c);
e) drop casting a nanocomposite on the active region of the C ink-coated WE of step (c);
f) drop casting ISM on the nanocomposite-coated active region of WE of step (e);
g) spray depositing single-walled carbon nanotubes (SWCNT) on top of the Ag/AgCl-coated RE of step (d);
h) drop casting of RE membrane on top of the SWCNT coated RE of step (g); and
i) forming a passivation layer on the connection region of the WE and the RE.
   59. The method of any one or combination of items herein, wherein the nanocomposite is drop-casted on the WE using a robotic spray dispenser.
   60. The method of any one or combination of items herein, wherein the nanocomposite comprises a alkylthiophene and a transition metal dichalcogenide.
   61. The method of item 60, wherein the alkylthiophene is poly(3-octyl-thiophene) (PoT).
   62. The method of any one or combination of items herein, wherein the transition metal dichalcogenide is molybdenum disulfide (MoS₂).
   63. The method of any one or combination of items herein, wherein said nanocomposite is prepared by mixing PoT with MoS₂ in a polar aprotic solvent to make a mixture.
   64. The method of item 63, wherein said polar aprotic solvent is tetrahydrofuran (THF).
   65. The method of any one or combination of items herein, wherein the mixture is further (1) magnetically stirred the mixture at 600-1000 rpm for at least 20 minutes to make a solution; and (2) horn sonicated at 30-50% intensity for at least 20 minutes.
   66. The method of any one or combination of items herein, wherein the ISM comprises a organophosphorus compound, a cellulose compound, an organic solvent, a high-strength thermoplastic material, an ionic surfactant and a polar aprotic solvent.
   67. The method of item 66, wherein the ISM is prepared by mixing the organophosphorus compound, the cellulose compound, the an organic solvent, the high-strength thermoplastic material, and the ionic surfactant in the polar aprotic solvent.
   68. The method of any one or combination of items herein, wherein the organophosphorus compound is methyltriphenylphosphonium bromide.
   69. The method of any one or combination of items herein, wherein the cellulose compound is nitrocellulose.
   70. The method of any one or combination of items herein, wherein the organic solvent is 2-nitrophenyl octyl ether.
   71. The method of any one or combination of items herein, wherein the high-strength thermoplastic material is polyvinyl chloride (PVC).
   72. The method of any one or combination of items herein, wherein the ionic surfactant is tridodecylmethylammonium nitrate.
   73. The method of any one or combination of items herein, wherein the polar aprotic solvent is tetrahydrofuran (THF).
   74. The method of any one or combination of items herein, wherein the SWCNT are spray-coated on the RE using a robotic dispenser over the electrode surface 50-150 times at a bed temperature of 50-90° C.
   75. The method of any one or combination of items herein, wherein said SWCNT are prepared by (1) making a solvent by mixing SDS with deionized (DI) water, (2) stirring the solvent of (1), (3) adding SWCNT to the solvent of (2) to make a solution, (4) mixing the solution of (3).
   76. The method of any one or combination of items herein, wherein said SWCNT are prepared by (1) making a solvent by mixing 250 mg of 0.5% SDS with deionized (DI) water, (2) stirring the solvent of (1) for 1 hour at 600 rpm, (3) adding 25 mg of 0.05% SWCNT to the solvent of (2) to make a solution, (4) stirring the solution of (3) for 30 minutes, (5) horn-sonicating the solution of (4) an ice-filled beaker for 30 minutes at 50% intensity, and (6) centrifuging the solution of (5) at 13,200 rpm for 110 minutes.
   77. The method of any one or combination of items herein, wherein the RE is first coated with the SWCNT and then coated with a RE membrane.
   78. The method of item 77, wherein the RE membrane comprises a resin, NaCl and methanol.
   79. The method of item 78, wherein the resin is poly(vinyl butyral) (PVB).
   80. The method of any one or combination of items herein, wherein the RE membrane is prepared by (1) preparing a solution by dissolving poly(vinyl butyral) (PVB) and NaCl in methanol, and (2) sonicating in an ice bath.
   81. The method of any one or combination of items herein, wherein the substrate is 0.5-10 cm in vertical length.
   82. The method of any one or combination of items herein, wherein the substrate is 1-4 cm in vertical length.
   83. The method of any one or combination of items herein, wherein the substrate includes a cone tip configured to penetrate a soil.
   84. The method of any one or combination of items herein, wherein the substrate is rectangular in shape.
   85. The method of any one or combination of items herein, wherein the substrate comprises a resin material.
   86. The method of item 85, wherein the resin material has a post-cured tensile strength of greater than 40 MPa.
   87. The method of any one or combination of items herein, wherein the resin material has a post-cured flexural strength of greater than 60 MPa.
   88. The method of any one or combination of items herein, wherein the resin material has a post-cured notched izod impact strength of greater than 35 J/m.
   89. The method of any one or combination of items herein, wherein the resin material has a post-cured heat deflection temperature at 1.8 MPa of between 50-55° C.
   90. The method of any one or combination of items herein, wherein the resin material has a post-cured heat deflection temperature at 0.45 MPa of between 60-65° C.
   91. The method of any one or combination of items herein, wherein the resin material has a post-cured thermal expansion of between 85-95 μm/m/° C. at 0-150° C.
   92. The method of any one or combination of items herein, wherein the two grooves on the front surface are between 0.5-10 mm in diameter.
   93. The method of any one or combination of items herein, wherein the two grooves on the front surface are between 1-2 mm in diameter.
   94. The method of any one or combination of items herein, wherein the two grooves on the front surface are between 0.4-1 mm in depth.
   95. The method of any one or combination of items herein, wherein the active region is spherical in shape.
   96. The method of any one or combination of items herein, wherein the active region is between 2.5-3.5 mm in radius.
   97. The method of any one or combination of items herein, wherein the active region is between 0.4-0.7 mm in depth.
   98. The method of any one or combination of items herein, wherein the RE membrane coated RE is resistant to chlorine leaching.
   99. A method for making a real-time monitoring system for in situ soil nitrate concentration analysis comprising:
a) making at least one potentiometric soil sensor comprising the steps of:
   i) 3D printing a substrate including at least two grooves on a front surface and two grooves in the back surface transversing from one end of the substrate to the other end of the substrate along a vertical direction;
   ii) inserting a WE comprising an active region and a connection region and a RE comprising an active region and a connection region into the grooves of the substrate;
   iii) brush coating the WE and the RE with C ink;
   iv) brush coating Ag/AgCl ink on the C ink-coated RE of step (iii);
   v) drop casting a nanocomposite on the active region of the C ink-coated WE of step (iii);
   vi) drop casting ISM on the nanocomposite-coated active region of WE of step (v); vii) spray depositing SWCNTs on top of the Ag/AgCl-coated RE of step (iv);
   viii) drop casting of RE membrane on top of the SWCNT coated RE of step (vii); and
   ix) forming a passivation layer on the connection region of the WE and the RE;
b) making at least one connector; and
c) mechanically attaching the least one potentiometric soil sensor to the at least one connector.
   100. The method of item 99, wherein the nanocomposite is drop-casted on the WE using a robotic spray dispenser.
   101. The method of any one or combination of items herein, wherein the nanocomposite comprises an alkylthiophene and a transition metal dichalcogenide.
   102. The method of any one or combination of items herein, wherein the nanocomposite comprises poly(3-octyl-thiophene) and molybdenum disulfide (PoT-MoS₂).
   103. The method of any one or combination of items herein, wherein said nanocomposite is prepared by mixing the alkylthiophene and transition metal dichalcogenide.
   104. The method of any one or combination of items herein, wherein said nanocomposite is prepared by mixing PoT with MoS₂ in a polar aprotic solvent to make a mixture.
   105. The method of item 104, wherein said polar aprotic solvent is tetrahydrofuran (THF).
   106. The method of any one or combination of items herein, wherein the mixture is further (1) magnetically stirred to make a solution; and (2) sonicated.
   107. The method of any one or combination of items herein, wherein the ISM comprises an organophosphorus compound, a cellulose compound, an organic solvent, a high-strength thermoplastic material, an ionic surfactant and a polar aprotic solvent.
   108. The method of item 107, wherein the organophosphorus compound is methyltriphenylphosphonium bromide, the cellulose compound is nitrocellulose, the organic solvent is 2-nitrophenyl octyl ether, the high-strength thermoplastic material is polyvinyl chloride, the ionic surfactant is tridodecylmethylammonium nitrate and the polar aprotic solvent is THF.
   109. The method of any one or combination of items herein, wherein the ISM is prepared by mixing an organophosphorus compound, a cellulose compound, an organic solvent, a high-strength thermoplastic material, an ionic surfactant and a solvent.
   110. The method of any one or combination of items herein, wherein the ISM is prepared by mixing methyltriphenylphosphonium bromide, nitrocellulose, 2-nitrophenyl octyl ether, polyvinyl chloride, and tridodecylmethylammonium nitrate in a solvent, optionally wherien the solvent is a polar aprotic solvent, and optionally wherein the solvent is THF.
   111. The method of any one or combination of items herein, wherein the SWCNT are spray-coated on the RE using a robotic dispenser over the electrode surface at least 80 times at a bed temperature of 50-90° C.
   112. The method of any one or combination of items herein, wherein said SWCNT are prepared by (1) making a solvent by mixing SDS with deionized (DI) water, (2) stirring the solvent of (1) for at least 30 minutes at 400-800 rpm, (3) adding 10-40 mg of 0.03-0.1% SWCNT to the solvent of (2) to make a solution, (4) mixing the solution of (3).
   113. The method of any one or combination of items herein, wherein said SWCNT are prepared by (1) making a solvent by mixing 200-300 mg of 0.03-0.1% SDS with deionized (DI) water, (2) stirring the solvent of (1) for 45-75 min at 400-800 rpm, (3) adding 20-30 mg of 0.05% SWCNT to the solvent of (2) to make a solution, (4) stirring the solution of (3) for 20-40 minutes, (5) horn-sonicating the solution of (4) an ice-filled beaker for 20-40 minutes at 40-60% intensity, and (6) centrifuging the solution of (5) at 12,000-14,000 rpm for 100-120 minutes.
   114. The method of any one or combination of items herein, wherein the RE is first coated with the SWCNT and then coated with a RE membrane.
   115. The method of any one or combination of items herein, wherein the RE comprising resin, NaCl and methanol.
   116. The method of any one or combination of items herein, wherein the RE membrane is prepared by (1) preparing a solution by dissolving poly(vinyl butyral) (PVB) and NaCl in a solvent, and (2) sonicating.
   117. The method of any one or combination of items herein, wherein the real-time monitoring system comprises the following structural assembly in a top to bottom direction: [C1]-[S1]-[C2]-[S2]-[C3]-[S3], wherein C1 is a first connector, S1 is a first potentiometric sensor, C2 is a second connector, S2 is a second potentiometric sensor, C3 is a third connector, S3 is a third potentiometric sensor.
   118. The method of any one or combination of items herein, wherein the real-time monitoring system further comprises at least one joining part, said joining part structurally reinforcing the mechanical attachment of each connector to each potentiometric sensor.
   119. The method of any one or combination of items herein, wherein the substrate comprises a resin material.
   120. The method of item 119, wherein the resin material has a post-cured tensile strength of at least 40 MPa or 40-50 MPa.
   121. The method of any one or combination of items herein, wherein the resin material has a post-cured flexural strength of at least 60 MPa or 60-70 MPa.
   122. The method of any one or combination of items herein, wherein the resin material has a post-cured notched izod impact strength of at least 35 J/m or 35-50 J/m.
   123. The method of any one or combination of items herein, wherein the resin material has a post-cured heat deflection temperature at 1.8 MPa of between 50-55° C.
   124. The method of any one or combination of items herein, wherein the resin material has a post-cured heat deflection temperature at 0.45 MPa of between 60-65° C.
   125. The method of any one or combination of items herein, wherein the resin material has a post-cured thermal expansion of between 85-95 μm/m/° C. at 0-150° C.
   126. The method of any one or combination of items herein, wherein the two grooves on the front surface are between 1-2 mm in diameter.
   127. The method of any one or combination of items herein, wherein the two grooves on the front surface are between 0.4-1 mm in depth.
   128. The method of any one or combination of items herein, wherein the active region is spherical in shape.
   129. The method of any one or combination of items herein, wherein the active region is between 2.5-3.5 mm in radius.
   130. The method of any one or combination of items herein, wherein the active region is between 0.4-0.7 mm in depth.
   131. The method of any one or combination of items herein, wherein the potentiometric soil sensor is 1-4 cm in vertical length.
   132. The method of any one or combination of items herein, wherein the potentiometric soil sensor is 2 cm in vertical length.
   133. The method of any one or combination of items herein, wherein the C1 is 3 cm in vertical length, C2 is 2 cm in vertical length, and C3 is 2 cm in vertical length.
   134. The method of any one or combination of items herein, wherein the real-time monitoring system provides multi-depth nitrate concentration measurements.
   135. The method of any one or combination of items herein, wherein the each potentiometric soil sensor provides a different soil depth nitrate concentration measurement.
   136. The method of any one or combination of items herein, wherein the real-time monitoring system detects soil nitrate concentration at 5 cm, 10 cm, and 15 cm soil depth.
   137. The method of any one or combination of items herein, wherein the real-time monitoring system further comprises a data acquisition circuit.
   138. The method of item 137, wherein the data acquisition circuit comprises an analog front end (AFE), a microcontroller unit (MCU), and a data transmission circuit.
   139. The method of item 138, wherein the AFE receives the soil nitrate concentration measurement from the RE and WE.
   140. The method of any one or combination of items herein, wherein the real-time monitoring system further comprises a wireless transmitter for transmitting the soil nitrate concentration measurement signal to a remote location.
   141. The method of item 140, wherein the wireless transmitter is an integrated dual-band transceiver antenna system.
   142. The method of any one or combination of items herein, wherein the wireless transmitter comprises two frequency bands.
   143. A method of selectively measuring nitrate in soil, comprising:
inserting a probe comprising at least one potentiometric soil sensor into a soil; generating an electrical potential between a reference electrode (RE) and a working electrode (WE), wherein the potential is based on nitrate levels in the soil; producing an electrical signal in response to the electrical potential between the RE and the WE, wherein said electrical signal indicates the soil nitrate concentration; transmitting the electrical signal to a data acquisition circuit to apply a calibration operation; determining the soil nitrate concentration based on the calibration operation.
   144. The method of item 143, wherein the probe contains a plurality of potentiometric sensors.
   145. The method of any one or combination of items herein, wherein the probe further comprises at least one wireless transmitter capable of transmitting the soil nitrate concentration measurement signal to a remote location.
   146. The method of item 145, wherein the wireless transmitter is configured to transmit soil measurement data from each of the plurality of potentiometric sensors.
   147. The method of any one or combination of items herein, wherein the data acquisition circuit comprises an analog front end (AFE), a microcontroller unit (MCU), and a data transmission circuit.
   148. The method of any one or combination of items herein, wherein the data acquisition circuit applies at least one reference voltage during the calibration operation, the reference voltage providing a reference against which the potential measured by the RE and WE is compared.
   149. The method of any one or combination of items herein, wherein the electrical potential generated by the RE and WE ranges from −100 mV to +500 mV in response to the soil nitrate concentration.
   150. The method of any one or combination of items herein, wherein the probe continuously monitors the soil nitrate concentrations.
   151. The method of any one or combination of items herein, wherein the probe continuously monitors the soil nitrate concentrations at a frequency recorded between once a second to once a minute.
   152. The method of any one or combination of items herein, wherein the probe continuously monitors the soil nitrate concentrations at a frequency recorded between once a second to once a day.
   153. The method of any one or combination of items herein, wherein the probe continuously monitors the soil nitrate concentrations at a frequency recorded between once a second to once an hour.
   154. The method of any one or combination of items herein, wherein the probe provides multi-depth nitrate concentration measurements.
   155. The method of any one or combination of items herein, wherein the RE is coated in a thermoplastic resin.
   156. The method of item 155, wherein the thermoplastic resin reduces signal drift and improves sensitivity of the RE compared to a RE that is not coated in the thermoplastic resin.
   157. The method of item 156, wherein the thermoplastic resin reduces chloride ion leaching compared to a RE that is not coated in the thermoplastic resin.

### EXAMPLES

### Example 1: Exemplary Process for Making a Real-Time Monitoring System of the Disclosure

A Form 3B 3D printer was used for printing the 3-D printed platform (“substrate”), the connector and the joining part. As shown in FIG. 1, steps (a)-(h), the 3-D printed platform was printed using Tough 2000 resin, featuring two 1.5 mm grooves on the front surface, designated for the working electrode (WE) and the reference electrode (RE). The active regions of both electrodes were designed in a circular shape with a radius of 3.09 mm and a groove depth of 0.6 mm.

After printing the substrate, a brush printing method was applied to coat the conductive layers on the electrodes in step (b). Initially, carbon (C) ink was brush-coated on both the WE and RE, followed by an additional brush coating of Ag/AgCl ink over the C ink on the RE shown in step (c). Subsequently, a nanocomposite of poly(3-octyl-thiophene) and molybdenum disulfide (PoT-MoS₂) was drop-casted on the WE as shown in step (d) to facilitate ion-to-electron transduction with high hydrophobicity and redox properties. This nanocomposite was prepared by mixing 20 mg of PoT with 80 mg of MoS₂ in 6 mL of THF. The mixture was magnetically stirred at 800 rpm for 30 minutes. Next, the solution was horn sonicated at 40% intensity for 30 minutes, and spray deposited on top of the WE using a Nordson EFD, USA robotic spray dispenser. To make the sensor only selective to nitrate ion, a nitrate ion-selective membrane (ISM) was used. To prepare the nitrate ISM, 12.5 mg methyltriphenylphosphonium bromide, 96.5 mg nitrocellulose, 1.73 ml 2-nitrophenyl octyl ether, 470 mg polyvinyl chloride, and 125 mg tridodecylmethylammonium nitrate were mixed in THF. A 20 μL drop of ISM solution was then applied over the POT-MoS₂ coating on the WE as shown in step (e).

The RE was spray-coated with single-walled carbon nanotubes (SWCNTs) as shown in step (f). The SWCNT was selected for this use for its hydrophobicity and stable potential production characteristics and to increase the surface area of the RE. The SWCNTs solution was prepared by mixing 250 mg of 0.5% SDS with DI water, stirring for 1 hour at 600 rpm, subsequently adding 25 mg of 0.05% SWCNTs, and stirring for 30 minutes. The mixture was horn-sonicated in an ice-filled beaker for 30 minutes at 50% intensity and later centrifuged at 13,200 rpm for 110 minutes to remove unexfoliated nanotubes. This solution was then spray deposited onto the RE by passing the nozzle of Nordson's robotic dispenser over the electrode surface 100 times at a bed temperature of 70° C. To prevent chloride ion (Cl⁻) leaching, another membrane was formulated and drop-cast on the RE in step (g). This RE membrane was prepared by dissolving 1.58 g of poly(vinyl butyral) (PVB) and 1.00 g of NaCl in 20 milliliters of methanol, sonicated in an ice bath for 30 minutes, and then drop-casted on top of the SWCNT coating in three 10 μL increments, totaling 30 μL.

Finally, as shown in step (h), a passivation layer was applied to both electrodes, extending from the bottom edge of the circular area to the electrode traces. This passivation layer ensures accurate and reliable signal generation by preventing the analyte solution from short circuiting the WE and RE connections.

Following the fabrication of the individual nitrate sensors, a sensor array was assembled to form the real-time monitoring system, as shown in FIG. 2 steps (a)-(f). This aspect of the real-time monitoring system of the disclosure enables nitrate measurements at soil depths of 5, 10, and 15 cm. Several components were first 3D printed using PLA filament then combined with the three nitrate sensors to realize the final structure. At first, the sensor designated for nitrate measurement at a depth of 15 cm (shown in step (a)) was attached to a 2 cm long connector (shown in step (b)). Next, the other end of the 2 cm connector was attached to the sensor designated for 10 cm depth (shown in step (c)). As shown in step (d) another 2 cm connector was then added, serving as a bridge between the sensors intended for 5 cm and 10 cm depths (as shown in step (e)). As shown in step (f), a 3 cm connector was finally attached to complete the sensor array for measuring nitrate at three soil depths. This assembly included three sensors (one for each depth-5 cm, 10 cm, and 15 cm), two 2 cm connectors, one 3 cm connector, five joining parts, and 20 screws (g), all integrated into a single probe-like structure.

FIGS. 3A-3B show the final structure following the assembly of the 3D printed parts. Each sensor features two 0.6 mm deep grooves for WE and RE. As shown in FIG. 3A, each connector has two 0.6 mm deep grooves on the front to route the wires, while the rear surface (shown in FIG. 3B) of the sensor array also has grooves to pass the wires through. Each of the three sensors has two wires for the WE and RE, making a total of six wires. As shown in FIG. 3B, these were routed through two 3 mm wide grooves at the back, which easily accommodated the 0.5 mm diameter wires.

### Example 2: Data Acquisition Circuit

As shown in FIGS. 4A-4B, The data acquisition circuit consists of an analog front end (AFE), a microcontroller unit (MCU), and a data transmission circuit. The sensors generate potential differences ranging from −100 mV to +500 mV between the reference electrode (RE) and the working electrode (WE) in response to nitrate ion concentrations. The internal resistance of the sensors can vary significantly, typically between hundreds of kilo ohms to tens of mega ohms, depending on soil conditions. These characteristics necessitate a precision, low-noise analog front end with high input impedance. The INA333, a zero-drift, rail-to-rail output instrumentation amplifier from Texas Instruments, was chosen for this application. The chip primarily aims to provide the required gain without imposing a loading effect.

A band-gap-based precision voltage reference from Texas Instruments, the REF2030, was selected to provide a stable voltage reference for the system. It offers an initial accuracy of 0.05% and low long-term drift of 8 ppm/° C. This reference chip provides both 1.5V and 3V simultaneously. The 1.5V reference was used to offset the signal voltage, enabling the measurement of negative input voltages using the single supply analog-to-digital converter (ADC) within the microcontroller. The 3V reference serves as the voltage reference for the internal 12-bit ADC. A first-order passive lowpass filter (LPF) with a cutoff frequency of 100 Hz was introduced after the amplifier to attenuate any disturbance or picked-up noise. The AFE, comprising three identical amplifier-filter circuits and the voltage reference, interfaces the three-sensor sensor array to the microcontroller. A set of shielded twisted-pair cables connect the sensors' RE and WE terminals with the analog front end, reducing noise pick-up.

The STM32L432 is an ultra-low-power microcontroller from STMicroelectronics and was chosen as the main processor due to its powerful 32-bit ARM Cortex-M4 core and extensive peripheral set. The integrated 12-bit ADC combined with the 3V external voltage reference allows the digitization of the input signal with a resolution of approximately 732.42 V. The firmware for this microcontroller is structured around a finite state machine (FSM). The ADC conversions are triggered by a timer-interrupt, after which the FSM processes the digitized data and transmits it via the on-board universal asynchronous receiver transmitter (UART) serial communication system to an external UART-to-USB chip.

### Example 3: Real-Time Monitoring System Application

The real-time monitoring system made using the process described in Example 1 (“the real-time monitoring system”) was employed to measure nitrate concentrations in three plant species (corn, cowpea and tomato) at three soil depths (5 cm, 10 cm and 15 cm). Soil moisture levels were carefully regulated, ranging from 20% to 50%, with water added incrementally to adjust moisture content. The main objective was to analyze nitrate fluctuations in the soil of three plant species under varying moisture conditions. Additionally, the soil nitrate dynamics surrounding the plant roots provided insights into how soil moisture levels and plant root architecture affect nitrate uptake by plants.

### Sensor Calibration

For this study, the real-time monitoring system was calibrated using actual soil samples to establish accurate reference points for its performance. The calibration process involved preparing various nitrate-loaded soil samples by adding a nitrate stock solution to soil-water mixtures. Soil samples were sun-dried for 5 hours, after which 50 grams of dried soil were mixed with 25 mL of water in separate containers. A 3000-ppm nitrate solution was prepared, and small volumes were gradually added to the soil-water mixtures to achieve target nitrate concentrations of 5, 9, 12, 25, 47, 68, and 90 ppm, which were confirmed using a commercial nitrate sensor (Horiba LAQUAtwin Model NO3-11). FIGS. 5A-5B shows the calibration results obtained with 3 sensors. For each nitrate concentration, the sensor response was recorded continuously for 2 minutes (FIG. 5A). The average value from the steady voltage readings during this period was recorded, and the voltage value corresponding to each nitrate concentration was plotted to produce the calibration plot (FIG. 5B).

The sensor was also calibrated for sensitivity. As shown in FIG. 5C, the sensitivity of a sensor of the disclosure was found to be −93.63 mV/(Log, ppm) and limit of detection (LOD) was calculated as 4.6297 ppm. To calculate the sensitivity, the voltage response of the sensor was plotted against the logarithm of nitrate concentrations, showing a linear relationship. The slope of this linear fit was used as a measure of sensitivity. The LOD was determined using the following equations:

Limit of Blank (LOB): LOB=mean of signal(blank sample)+1.645*(std.dev.of blank sample) Intermediate value for Limit of Detection (yLOD): yLOD=LOB+1.645*(std.dev.of target at low concentration)

### Limit of Detection (LOD):

$$LOD = \frac{yLOD - intercept}{slope}$$

### Longevity Test

For the longevity test, three real-time monitoring systems were buried in the soil at a depth of 5 cm, and real-time monitoring system response was recorded 3 times a day (10 AM, 2 PM, 6 PM). The average reading from the three systems at each time point was calculated. The results plotted in FIG. 6A show that the systems remained functional for up to 7 days without the need for any recalibration.

Starting from day 8, system performance started to decline, which could be attributed to reduced stability of the reference electrode, requiring recalibration of the systems on day 8. Data was not collected beyond day 10. Physical factors like abrasion and mechanical stress can disrupt the ion-selective membrane coating on the real-time monitoring systems of the disclosure. Regular handling, such as inserting or removing the real-time monitoring systems from samples, can also degrade the coating, leading to cracks or scratches that compromise their integrity. Additionally, repeated immersion in different soil samples may cause friction against the real-time monitoring system's surface, contributing to wear. These factors ultimately reduce the real-time monitoring systems' sensitivity and selectivity, affecting their accuracy in nitrate measurements. The nitrate concentrations measured with the real-time monitoring system were verified against a commercial sensor (LaquaTwin) and the measurements at each time point were comparable. The maximum and minimum concentration found from the sensor was 132 PPM at noon on day 8 and 53 PPM on day 9.

### Selectivity Test

In order to evaluate the selectivity of the real-time monitoring system of the present disclosure, it was tested for nitrate ions in the presence of potential interfering ions. Phosphate, sulfate, sodium, magnesium and chloride salts were selected due to their abundance in the soil. First, 1000 ppm solutions of these ions were prepared, and the real-time monitoring system readings were recorded in the presence of these ions alone. Following that, the real-time monitoring system reading was taken for a 1000-ppm nitrate solution. Finally, the real-time monitoring system was immersed in a solution containing both 1000 ppm nitrate and a 1000-ppm concentration of an interfering ion. The sensor response in only nitrate solution was 244.167 mV.

In order to evaluate the selectivity of the ion-selective electrodes (ISEs) of the real-time monitoring systems of the disclosure in the presence of interfering ions, the Fixed Primary Ion Method (FPM) technique was used for selectivity analysis. The FPM quantifies the response of an ISE to an interfering ion relative to its response to the primary ion.

The ions in the soil are: phosphate (PO₄²⁻), sulfate (SO₄²⁻), magnesium (Mg²⁺), nitrite (NO₂⁻), and chloride (Cl⁻) salts. First, 1000 ppm solutions of these ions were prepared in DI water, and the sensor readings were recorded in the presence of these ions alone. Following that, the sensor reading was taken for a 1000-ppm nitrate solution. Finally, the sensor was immersed in a solution containing 1000 ppm nitrate and 1000 ppm concentration of an interfering ion. The sensor response in only nitrate solution was 244.167 mV.

By evaluating the sensor's response in a range of solutions that include the target ion along with a variety of interfering ions, this method makes it possible to compare possible results and ascertain the impact of each ion on the sensor's functionality. This influence is measured by the k coefficient calculated using the equation:

$$k = \frac{{α}_{A}}{{α}_{B}^{\frac{{z}_{A}}{{z}_{B}}}} ;$$

a larger k value denotes severe interference, whereas a value near 1 denotes little effect. The k coefficient is crucial for describing the selectivity of a sensor since it aids in forecasting the performance of samples containing numerous ions in real-world scenarios and guides calibration techniques to improve measurement precision. As shown in FIG. 6B, the real-time monitoring system is comparatively susceptible to nitrate and chloride ions compared to other interfering ions.

### Cyclic Test

The real-time monitoring system was subjected to progressively increasing and then decreasing concentrations of nitrate over four cycles, as illustrated in FIG. 6C. For this test, the real-time monitoring system was placed in soil samples with nitrate concentrations of 9, 25, 47, 68, and 90 ppm, with each concentration tested eight consecutive times. Throughout these cycles, the real-time monitoring system consistently displayed stable responses to each nitrate concentration, with a standard deviation of less than 5.6%. This performance highlights the real-time monitoring system's reliability and precision in detecting varying nitrate levels. Additionally, the low deviation underscores the real-time monitoring system's robustness, making it suitable for applications requiring accurate, repeatable measurements over extended periods.

### Soil Moisture Test

To assess the effect of soil moisture on the real-time monitoring system's performance, the real-time monitoring system was calibrated as described in the section above and repeated for various moisture levels, including 10%, 20%, 30%, 40%, and 50%. Each moisture condition was carefully controlled to mimic soil conditions ranging from dry to wet. Soil samples with nitrate concentrations of 5, 9, 12, 25, 47, 68, and 90 ppm were prepared for each moisture level. The real-time monitoring system's response was recorded continuously for 2 minutes at each nitrate concentration. As shown in FIG. 6D, fluctuations in real-time monitoring system response were observed at 10% moisture. However, the real-time monitoring system of the disclosure shows reliable performance starting at 20% of soil moisture level.

### Example 4: Dynamic Test Results

The inventors tested the real-time monitoring system's performance using three plant species—corn, cowpea, and tomato—each with two replicates, as well as one instance of bare soil. The developed sensor array was buried in the soil of these potted plants to monitor nitrate levels in real-time at depths of 5, 10, and 15 cm. Initially, the soil moisture level was set to 20% at a depth of 10 cm (time, t=0). Nitrate concentrations were then measured continuously at all three depths for 1 hour. After the first hour, the recorded moisture level at 10 cm showed a slight variation from the initial setting for all plants.

Subsequently, the moisture level was increased incrementally by 5% at 10 cm depth every hour over a period of 5 hours. After each adjustment in moisture level, nitrate measurements were taken continuously for one hour, resulting in a total of 5 hours of nitrate data collection from each plant. After the 5-hour monitoring period, the real-time monitoring system was removed, and the nitrate content in the soil was measured by the commercial sensor.

### Nitrate Measurements

FIGS. 7A-7I show the dynamic nitrate measurements in both bare soil after the plant was removed and in soil with the plant still present. In the bare soil after the corn plant was removed, labeled as “Corn Bare Soil” in FIG. 7A, the average nitrate concentration recorded over a 5-hour period was roughly 51 ppm at a depth of 25 cm and approximately 500 ppm at 5 cm. In “Corn Plant 1” (with the plant alive and present), the highest recorded nitrate concentration was 90 ppm at 15 cm, with a gradual decrease at shallower depths, shown in FIG. 7B. The average nitrate level for “Corn Plant 2” was approximately 60 ppm at a depth of 15 cm, shown in FIG. 7C.

For cowpea, in the bare soil after plant removal, labeled “Cowpea Bare Soil” in FIG. 7D, the nitrate level peaked at approximately 68 ppm at a depth of 10 cm after 1 hour and then declined. The nitrate concentration demonstrated a decreasing trend as the soil depth decreased (FIGS. 7E and 7F). For both “Cowpea Plant 1” and “Cowpea Plant 2”, nitrate concentrations at 10 and 15 cm depths were quite similar (FIG. 7F). This was further confirmed with the commercial LaquaTwin probe. In 10 cm of soil depth, the most concentration was measured 71 PPM, the lowest at 39 PPM, and in 15 cm of depth, the highest at 69 PPM and the lowest at 45 PPM.

Tomato plants demonstrated the opposite trend, with nitrate concentrations measured by the real-time monitoring system as increasing as soil depths decreased, as shown in FIGS. 7G, 7H, and 7I. Moreover, nitrate concentrations in all tomato plants remained below 25 ppm across all depths. These results could be attributed to the differences in root structures among the different plant species, as discussed below.

### Root Architecture Effects on Soil Nitrate Levels

Corn develops a shallow root system during its early growth stages, which supports the plant's establishment and initial growth. In such plants, the topsoil serves as the primary source of nitrate. If the topsoil has a high nitrate concentration, shallow-rooted plants can utilize it effectively. However, since their roots do not easily access deeper soil layers where nitrate may accumulate, they are more susceptible to the conditions of the surface soil. The corn plants used in this study were small and still growing, with lateral roots reaching 7 cm and vertical roots extending 6 cm, as shown in FIG. 8A. As a result, these plants primarily absorbed nitrate from the upper soil, at around 5 cm depth. Consequently, the nitrate levels were measured by the real-time monitoring system to be lower at 5 cm compared to the 15 cm depth, where a higher concentration of nitrate was detected. The elevated nitrate levels at 15 cm also suggest that nitrate leaching occurred through the soil layers. These findings are in agreement with the results presented in the previous section.

In the early growth stages, cowpea develops a taproot that descends vertically and serves as the primary root from which lateral roots grow. This taproot provides structural stability and helps anchor the plant in the soil. Unlike fibrous roots, the taproot can penetrate deeper soil layers, accessing water and nutrients that may not be available in the upper layers, especially during dry periods. In well-drained soils, the cowpea's narrow taproot allows the plant to reach deeper moisture and nutrients. However, in shallow or compacted soils, the fibrous roots play a more crucial role in nutrient absorption, as the taproot may not extend as far. The small cowpea plant used in this experiment was grown in a pot did not have optimal conditions to develop fully. Consequently, the roots primarily drew nutrients from the upper soil layers, leading to a reduction in nitrate levels at a depth of 5 cm as measured by the real-time monitoring system of the disclosure. As shown in FIG. 8B, the cowpea's roots, which extended only 4 cm deep and wide and could not reach the deeper soil layers to absorb nutrients. As a result, nitrate concentrations measured at 10 and 15 cm depths were similar and higher than those at 5 cm depth. These results align with the trend in nitrate concentrations presented in the previous section.

During germination, the tomato plant initially develops a taproot, the primary root system that grows downward to anchor and support the plant during its early stages. As the plant matures, the dominance of the taproot diminishes, and a fibrous root system emerges. This fibrous root network spreads horizontally and can extend up to 90 cm or more, depending on soil conditions, allowing the plant to access a larger soil area for water and nutrients. The dense fibrous roots near the surface enhance nutrient and moisture absorption. The tomato plant used in this study had roots that extended 14 cm laterally and 22 cm vertically as shown in FIG. 8C. As the taproot extended deeper, the inventors measured a lower nitrate concentration at the 15 cm depth using the real-time monitoring system of the present disclosure compared to the surface soil. Also, the nitrate concentration at the 5 cm depth was also lower than in other plants, likely due to the extensive horizontal spread of the fibrous roots near the soil surface, which enabled higher nitrate absorption from the upper layers. Because the tomato plants were grown in loamy soil, it was able to retain water and create a more compact soil structure. These results agree with the results presented in the previous section.

### Soil Moisture Effects on Soil Nitrate Levels

Soil moisture levels significantly influence the amount of nitrate that plants can absorb. Adequate moisture enhances nitrate availability by dissolving and mobilizing nitrates within the soil, allowing plant roots to efficiently take up these nutrients, thereby promoting healthy growth and development. Conversely, excessive moisture can lead to waterlogging, which reduces oxygen levels in the root zone, inhibits root growth, and increases the risk of nitrate leaching. This results in decreased nitrate absorption and potential nutritional deficiencies. Similarly, insufficient water availability during drought conditions limits nitrate mobility, negatively impacting plant health and reducing yields by obstructing root growth and nitrate uptake. Therefore, balanced irrigation and effective moisture management are essential for optimizing nitrate uptake and ensuring overall plant health and productivity.

Table 1 shows all the moisture percentage reading in every plant described in the sections above at different depths at every hour, and the moisture level was increased after each hour by adding water to increase the moisture. Table 1 shows that the moisture level in the soil increased after each hour at all depths. At an increasing moisture level, the real-time monitoring system of the disclosure measured nitrate mobility increases. This suggests that the plant's root can easily absolve the nitrate and transport it to other parts. Also, at a high moisture level, the nitrate ion is leached downward, thus increasing the concentration of nitrate in the lower level of soil.

TABLE 1

| | Plant | | | | | | | | |
|---|---|---|---|---|---|---|---|---|---|
| | Corn Bare | | | Cowpea Bare | | | Tomato Bare | | |
| | Depth (cm) | | | | | | | | |
| Hour | 5 | 10 | 15 | 5 | 10 | 15 | 5 | 10 | 15 |
| 1 | 10.1 | 21.5 | 27.35 | 9.45 | 21.85 | 26.3 | 11 | 20.65 | 25.35 |
| 2 | 13.65 | 24.75 | 33.15 | 8.45 | 26.05 | 33.35 | 13.15 | 24.25 | 30.45 |
| 3 | 15.5 | 33.15 | 37.45 | 9.7 | 31.3 | 37.85 | 15.3 | 29.35 | 37.1 |
| 4 | 17.9 | 34.4 | 42.1 | 12.95 | 36.7 | 43.05 | 18.4 | 37.1 | 43.85 |
| 5 | 20.15 | 39.35 | 47.25 | 13 | 40.85 | 47.65 | 20.15 | 43.1 | 49.05 |
| | Plant | | | | | | | | |
| | Corn Plant 1 | | | Cowpea Plant 1 | | | Tomato Plant 1 | | |
| | Depth (cm) | | | | | | | | |
| Hour | 5 | 10 | 15 | 5 | 10 | 15 | 5 | 10 | 15 |
| 1 | 9.05 | 20.9 | 24.2 | 7.45 | 20.9 | 26.3 | 13 | 20.5 | 25.4 |
| 2 | 11.8 | 24.5 | 29.7 | 8.45 | 26.1 | 32.9 | 14.6 | 24.7 | 31.1 |
| 3 | 17.1 | 29.7 | 35.6 | 9.7 | 32.9 | 37.9 | 17.7 | 31.1 | 37.9 |
| 4 | 21.8 | 33.1 | 40.2 | 13 | 36.7 | 43.7 | 19 | 35.2 | 39.7 |
| 5 | 24.3 | 38.4 | 46.9 | 13 | 40.9 | 48.4 | 20.3 | 38.7 | 46 |
| | Plant | | | | | | | | |
| | Corn Plant 2 | | | Cowpea Plant 2 | | | Tomato Plant 2 | | |
| | Depth (cm) | | | | | | | | |
| Hour | 5 | 10 | 15 | 5 | 10 | 15 | 5 | 10 | 15 |
| 1 | 11.1 | 21.2 | 25 | 8.8 | 20.5 | 24.9 | 9.2 | 21 | 27.1 |
| 2 | 14.1 | 24.7 | 29.7 | 11.5 | 25.9 | 30.8 | 11.9 | 24.1 | 31.7 |
| 3 | 15.3 | 30.2 | 35.6 | 13.3 | 30.8 | 35.2 | 13.9 | 31.7 | 36.7 |
| 4 | 17.5 | 36.4 | 40.2 | 14.2 | 33.3 | 41 | 17.6 | 33.7 | 41.6 |
| 5 | 20.9 | 41.1 | 46.9 | 20.15 | 39.35 | 47.25 | 19.2 | 37.9 | 46.9 |

The measurements taken by the real-time monitoring system of the present disclosure were validated against a commercial sensor, which showed comparable results.

Nitrate levels were continuously monitored over a five-hour period, with water added to each pot at the end of every hour to raise moisture content by 5% at a depth of 10 cm. A commercial LaquaTwin sensor was used to measure nitrate levels at different depths at the 1st, 2nd, 3rd, 4th, and 5th hour mark. Nitrate concentrations recorded by the real-time monitoring system of the disclosure at these time points were validated against the LaquaTwin measurements. In the corn bare soil, nitrate levels increased with depth as more nitrate leached downward with increased moisture, as shown in FIG. 9A. A similar pattern was observed in the cowpea bare soil, as shown in FIG. 9D. In contrast, the tomato bare soil, being loamy with better water retention capability, showed less nitrate leaching to deeper soil, as shown in FIG. 9G.

In corn plant 1, as moisture increased, nitrate leached downward, but since the roots extended only to 6 cm, more nitrate accumulated below this depth, unable to be absorbed by the roots. As shown in FIG. 9B, at 5 cm, nitrate levels were lower due to root depletion. As shown in FIG. 9E, cowpea plant 1 exhibited a similar pattern, validated by the commercial LaquaTwin sensor. Tomato plant roots extended to 22 cm, and while moisture was highest in deeper soil, nitrate concentration was lowest there. As shown in FIG. 9H, higher nitrate levels were found near the surface, but as tomato has fibrous roots up to 14 cm, nitrate levels in the upper soil were comparatively higher than deeper levels. The loamy soil of the tomato plant, combined with greater nitrate uptake by the deeper tomato roots, led to a reduction in nitrate levels as soil depth increased.

As shown in FIGS. 9C and 9F, similar trends were seen in corn plant 2 and cowpea plant 2, resembling the results from corn plant 1 and cowpea plant 1, respectively. Likewise, as shown in FIG. 9I, the tomato plant 2 results agree with the plant 1 results.

FIGS. 10A-10C show regression plots, which compare nitrate measurements collected by the real-time monitoring system with LaquaTwin readings for all three plant species. The root mean square error (RMSE) values between the sensor and commercial LaquaTwin measurements as shown in FIGS. 10A-10C. For corn, the RMSE is 0.1125, while the RMSE is found to be 0.1038 for cowpea plants, indicating a high level of agreement between the two sensors.

In contrast, the tomato plant exhibited a higher RE of 0.2956. This inconsistency in measurements suggest potential challenges for the sensor in accurately and reliably measuring nitrate levels in certain plants, such as tomato plants. These discrepancies may stem from various factors, including lower nitrate concentrations in tomatoes, which are only slightly above the sensor's limit of detection (LOD) of 4.6297 ppm, or interference from the soil matrix.

### Example 5: F-Scores Under Different Soil Conditions

### F-Scores Across Soil Depths

The F-scores obtained from the analysis of variance (ANOVA) analysis of nitrate concentrations in soil at various depths provide key insights into the differences among plant types. As shown in FIG. 11A, the F-score for Corn Bare is significantly high at 22.92, highlighting substantial variation in nitrate levels across different soil depths. Other corn treatments show lower F-scores (10.89 and 15.77), yet still indicate notable variability in nitrate availability. In contrast, cowpea plants exhibit more moderate differences, with F-scores of 5.73 for Cowpea Bare, 11.28 for Cowpea Plant 1, and 12.16 for Cowpea Plant 2. While there is some variability in nitrate concentrations, it is not as pronounced as in corn. Lastly, the F-scores for tomato plants are relatively low, with values of 0.84, 1.27, and 2.38, indicating lower differences in nitrate concentrations across soil depths. Overall, these findings suggest that corn demonstrates the highest statistically significant difference in nitrate levels across different soil depths, while tomato exhibits a more uniform nitrate distribution.

### F-Scores Across Soil Moisture Levels

The F-scores across soil moisture levels indicate how moisture levels influence nitrate concentration at different soil depths for each plant. Moisture levels for all plants are shown in Table 1. As shown in FIG. 11B, Corn Bare (bare soil that previously held a corn plant) shows F-scores of 4.89 at 5 cm, 4.15 at 10 cm, and 1.42 at 15 cm, reflecting a statistically significant difference in the nitrate levels across moisture levels, with a higher variability at shallow depths. Corn Plant 1 has lower F-scores of 0.34, 1.29, and 1.17, at 5 cm, 10 cm, and 15 cm, respectively, suggesting relatively lower variability in nitrate levels, while Corn Plant 2 scores 0.91, 0.08, and 0.44, reflecting a weak response of nitrate levels to moisture changes, especially at 10 cm. In Cowpea plants, Cowpea Bare (soil that previously held a cowpea plant) has higher F-scores of 6.80, 3.28, and 4.91, particularly at 5 cm, showing strong moisture influence; Cowpea Plant 1 scores 1.62, 1.31, and 0.58, indicating decreasing responsiveness with increasing depth. Cowpea Plant 2 has F-scores of 4.64, 1.65, and 0.38, revealing a significant moisture effect at 5 cm but much less at greater depths. For Tomato, Tomato Bare scores 4.21, 1.92, and 3.09, indicating moderate responsiveness, while Tomato Plant 1 has F-scores of 3.29, 1.85, and 3.11, showing nitrate variability primarily at 5 cm. Lastly, Tomato Plant 2 scores 2.34, 2.01, and 3.19. Overall, in corn and cowpea plants, nitrate variability is higher as a function of soil depths, whereas tomato exhibits more stability in nitrate concentration across soil depths.

### F-Scores Across Plant Species

F-scores were computed to reflect the variability in nitrate levels among different plants-corn, cowpea, and tomato at varying soil depths. As shown in FIG. 11C, for bare soil at 5 cm, the F-score is 6.33, indicating a moderate level of variability in the nitrate concentrations among the three plants. This suggests that, there are differences in how each plant responds to the available nitrate at a shallow depth. At 10 cm, the F-score drops to 5.45, suggesting a decrease in variability among the plants as the soil becomes deeper, which may indicate that the nitrate levels are becoming more uniform across plant types. However, at 15 cm, the F-score jumps to 39.48, revealing substantial variability and indicating that the differences in nitrate levels among the plants become more pronounced at this depth, possibly due to deeper rooting patterns or nutrient uptake mechanisms. For Plant 1, the F-scores are 4.25 at 5 cm, 19.01 at 10 cm, and 36.78 at 15 cm, demonstrating an increasing trend in nitrate variability with increasing depth. This suggests that as the soil depth increases, the differences in nitrate levels among the plants become more significant. In contrast, Plant 2 shows F-scores of 2.70 at 5 cm, 34.44 at 10 cm, and 57.65 at 15 cm, indicating lower nitrate variability at the shallow depth but a significantly higher nitrate variability at greater depths compared to Plant 1.

### Example 6: Morphological Characterizations

### SEM Images

FIG. 12A and FIG. 12B present scanning electron microscopic (SEM) images illustrating the morphological characteristics of SWCNT and PoT-MoS2 layers, respectively. FIG. 12A shows the SWCNT layer, characterized by a network of interconnected fibrous structures forming a web-like morphology. In FIG. 12B, the PoT-MoS2 layer exhibits an irregular, densely packed granular morphology with layered and flake-like structures typical of MoS₂ and polymeric domains. This rough, compact texture suggests good interfacial adhesion between the PoT and MoS₂ components, potentially enhancing the layer's mechanical and electrical properties for applications in electronics or sensing.

### FTIR Analysis

As shown in FIG. 13A, the FTIR spectrum of the SWCNT dispersion prepared with SDS and DI water indicate specific functional groups. A broad peak at around 3300 cm⁻¹ suggests O—H stretching, likely from water molecules or hydrogen bonding within SDS, which may also indicate interactions between SDS and SWCNTs. Although a peak near 2900 cm⁻¹ is typically associated with C—H stretching in SDS's alkyl chains, it appears faint or absent in this FTIR spectrum, suggesting weak or overlapping C—H signals. A clear peak at around 1600 cm⁻¹ corresponds to C═C stretching, characteristic of the sp²-hybridized carbon bonds in SWCNTs. In the 1000-500 cm⁻¹ range, broad features are observed, likely due to S—O stretching from SDS and possibly due to C—O or C—N interactions with minor functional groups on the SWCNT surface, highlighting the SDS-SWCNT interactions within the dispersion.

As shown in FIG. 13B, the FTIR spectrum of the ISM shows several distinct peaks corresponding to the functional groups present at the membrane. A peak near 2900 cm⁻¹ suggests C—H stretching from the hydrocarbon chains present in compounds like nitrocellulose, 2-nitrophenyl octyl ether, and PVC. Peaks observed between 1600-1500 cm⁻¹ likely correspond to aromatic C═C stretching, possibly due to methyltriphenylphosphonium bromide or other aromatic structures within the solution. The range of 1250-1050 cm⁻¹ shows peaks associated with C—O and C—N stretching, which could originate from nitrocellulose and 2-nitrophenyl octyl ether, as well as potential C—Cl stretching from PVC. Additionally, peaks below 1000 cm⁻¹ might be due to P—C stretching from methyltriphenylphosphonium bromide and bending vibrations related to C—Cl from PVC, confirming the presence of the ISM components and their characteristic functional groups.

As shown in FIG. 13C, The FTIR spectrum of poly(octyl thiophene) (PoT) and molybdenum disulfide (MoS₂) demonstrates several peaks corresponding to the functional groups from each compound. A peak near 2900 cm⁻¹ corresponds to C—H stretching, likely from the octyl side chains in PoT and the THF solvent, both of which contain alkyl groups that contribute to this vibration. Around 1600 cm⁻¹, a distinct peak is observed, which can be attributed to C═C stretching in the conjugated thiophene rings of PoT, a characteristic feature of polythiophenes' delocalized electron systems. In the 1000-500 cm⁻¹ range, peaks appear that likely correspond to Mo—S stretching vibrations from MoS₂, which has distinctive stretching in this region. Additionally, C—S bonds from the thiophene rings of PoT may overlap here, further supporting the presence of both PoT and MoS₂ in the THE solution. This combination of peaks provides evidence of the primary bonds and functional groups present in each compound within the solution.

As shown in FIG. 13D, The FTIR spectrum of the RE membrane containing poly(vinyl butyral) (PVB) and NaCl in methanol show peaks that represent the functional groups of the components. A broad peak around 3400 cm⁻¹ corresponds to 0-H stretching, likely from the methanol solvent and any hydroxyl groups in PVB, indicating hydrogen bonding within the solution. Near 2900 cm⁻¹, a peak reflects C—H stretching, confirming the presence of alkyl groups in PVB and methanol. The strong peak around 1730 cm⁻¹ is indicative of C═0 stretching vibrations, a characteristic of the carbonyl groups in PVB's vinyl butyral units, while the region between 1100-1000 cm⁻¹ shows C—O stretching from both PVB and methanol

In addition, it is to be understood that any particular aspect of the present disclosure that falls within the prior art may be explicitly excluded from any one or more of the claims. Since such aspects are deemed to be part of the whole of the present disclosure, any part of the whole disclosure may be excluded even if the exclusion is not set forth explicitly herein.

It is to be understood that while the present disclosure has been described in conjunction with the detailed description thereof, the foregoing description is intended to illustrate and not limit the scope of the present disclosure, which is defined by the scope of the appended claims. Other aspects, advantages, and alterations are within the scope of the following claims.

1. A potentiometric soil sensor comprising:
(i) a substrate; and
(ii) a nitrate sensor comprising a nitrate ion-selective membrane (ISM) disposed on the substrate;
wherein the nitrate sensor comprises at least one working electrode (WE) and at least one reference electrode (RE);
wherein the WE and RE each comprise one or more conductive layers, and
wherein the WE and RE are disposed on a surface of the substrate.

2. The potentiometric soil sensor of claim 1, wherein the WE and the RE are each disposed in a separate groove on the surface of the substrate, and the WE and the RE each comprise a connection area and an active region.

3. The potentiometric soil sensor of claim 1, wherein the WE and RE comprise a carbon coating layer.

4. The potentiometric soil sensor of claim 3, wherein:
a) the RE further comprises a silver or silver chloride (Ag/AgCl) coating layer over the carbon coating layer;
b) the coating layers are a brush coated ink composition;
c) the RE further comprises a single-walled carbon nanotube (SWCNT) coating layer;
d) the WE further comprises a nanocomposite layer over the carbon coating layer, optionally the nanocomposite comprises poly(3-octyl-thiophene) and molybdenum disulfide (PoT-MoS₂);
e) the WE and RE comprise a passivation layer on at least part of the WE and RE, optionally the passivation layer is disposed on the connection area of the WE and RE; or
f) a combination of a)-e).

5. The potentiometric soil sensor of claim 1, wherein said substrate comprises:
a) a resin material with a post-cured tensile strength of greater than 40 MPa;
b) a resin material with a post-cured flexural strength of greater than 60 MPa;
c) a resin material with a post-cured notched izod impact strength of greater than 35 J/m;
d) a resin material with a post-cured heat deflection temperature at 1.8 MPa of between 50-55° C.;
e) a resin material with a post-cured heat deflection temperature at 0.45 MPa of between 60-65° C.;
f) a resin material with a post-cured thermal expansion of between 85-95 μm/m/° C. at 0-150° C. or
g) a combination of a)-f).

6. The potentiometric soil sensor of claim 2, wherein:
a) said grooves on the front surface are between 1-2 mm in diameter;
b) said grooves on the front surface are between 0.4-1 mm in depth;
c) said active region is spherical in shape;
d) said active region is between 2.5-3.5 mm in radius;
e) said active region is between 0.4-0.7 mm in depth; or
f) a combination of a)-e).

7. A probe comprising the potentiometric soil sensor of claim 1.

8. The probe of claim 7, comprising an array of the potentiometric soil sensors, wherein the array comprises at least two or at least three of the potentiometric sensors.

9. The probe of claim 8, wherein each potentiometric soil sensor is vertically stacked and mechanically attached to at least one connector.

10. The probe of claim 9, wherein each connector is positioned on top of each potentiometric soil sensor and wherein the active region of the WE and the active region of the RE are positioned away from the connector positioned on top of the potentiometric sensor.

11. The probe of claim 9, further comprising joining parts, said joining parts structurally reinforcing the mechanical attachment of each connector to each potentiometric sensor.

12. The probe of claim 9, comprising the following structural assembly in a top to bottom direction: [C1]-[S1]-[C2]-[S2]-[C3]-[S3], wherein C1 is a first connector, S1 is a first potentiometric sensor, C2 is a second connector, S2 is a second potentiometric sensor, C3 is a third connector, S3 is a third potentiometric sensor; wherein each connector is mechanically attached to each potentiometric soil sensor by a joining part; optionally wherein S3 includes a cone tip configured to penetrate soil.

13. The probe of claim 12, wherein:
a) each potentiometric soil sensor is 1-4 cm in vertical length, or about 2 cm in vertical length; or
b) C1 is about 3 cm in vertical length, C2 is about 2 cm in vertical length, and C3 is about 2 cm in vertical length.

14. The probe of claim 12, further comprising a data acquisition circuit comprising an analog front end (AFE), a microcontroller unit (MCU), and a data transmission circuit, wherein the AFE receives the soil nitrate concentration measurement from the RE and WE, optionally the AFE further comprises at least one instrumentation amplified (IN-AMP), at least one reference voltage and at least one lowpass filter (LPF).

15. The probe of claim 12, further comprising a wireless transmitter for transmitting the soil nitrate concentration measurement signal to a remote location, wherein the wireless transmitter is an integrated dual-band transceiver antenna system, optionally the wireless transmitter comprises two frequency bands.

16. A method for making at least one potentiometric soil sensor comprising the steps of:
a) 3D printing a substrate including at least two grooves on a front surface transversing from one end of the substrate to the other end of the substrate along a vertical direction;
b) disposing a WE comprising an active region and a connection region and a RE comprising an active region and a connection region into the two grooves on the front surface of the substrate;
c) brush coating the WE and the RE with carbon (C) ink;
d) brush coating Ag/AgCl ink on the C ink-coated RE of step (c);
e) drop casting a nanocomposite on the active region of the C ink-coated WE of step (c);
f) drop casting an ion selective membrane (ISM) on the nanocomposite-coated active region of WE of step (e);
g) spray depositing single-walled carbon nanotubes (SWCNT) on top of the Ag/AgCl-coated RE of step (d);
h) drop casting of RE membrane on top of the SWCNT coated RE of step (g); and
i) forming a passivation layer on the connection region of the WE and the RE.

17. The method of claim 16, wherein:
a) the nanocomposite is drop-casted on the WE using a robotic spray dispenser;
b) the nanocomposite comprises a alkylthiophene and a transition metal dichalcogenide, optionally the alkylthiophene is poly(3-octyl-thiophene) (PoT), optionally the transition metal dichalcogenide is molybdenum disulfide (MoS₂);
c) said nanocomposite is prepared by mixing PoT with MoS₂ in a polar aprotic solvent to make a mixture, optionally said polar aprotic solvent is tetrahydrofuran (THF);
d) a combination of a)-c).

18. The method of claim 16, wherein the ISM comprises a organophosphorus compound, a cellulose compound, an organic solvent, a high-strength thermoplastic material, an ionic surfactant and a polar aprotic solvent.

19. The method of claim 18, wherein:
a) the organophosphorus compound is methyltriphenylphosphonium bromide;
b) the cellulose compound is nitrocellulose;
c) the organic solvent is 2-nitrophenyl octyl ether;
d) the high-strength thermoplastic material is polyvinyl chloride (PVC);
e) the ionic surfactant is tridodecylmethylammonium nitrate;
f) the polar aprotic solvent is tetrahydrofuran (THF); or
g) a combination of a)-f).

20. The method of claim 16, wherein:
a) the SWCNT are spray-coated on the RE using a robotic dispenser over the electrode surface 50-150 times at a bed temperature of 50-90° C.; and/or
b) said SWCNT are prepared by (1) making a solvent by mixing SDS with deionized (DI) water, (2) stirring the solvent of (1), (3) adding SWCNT to the solvent of (2) to make a solution, (4) mixing the solution of.

21. The method of claim 16, wherein the RE is first coated with the SWCNT and then coated with a RE membrane.

22. The method of claim 21, wherein:
a) the RE membrane comprises a resin, NaCl and methanol, optionally the resin is poly(vinyl butaryl) (PVB);
b) the RE membrane is prepared by (1) preparing a solution by dissolving poly(vinyl butyral) (PVB) and NaCl in methanol, and (2) sonicating in an ice bath; and/or
c) the membrane coated RE is resistant to chlorine leaching.

23. The method of claim 16, wherein:
a) the substrate is 0.5-10 cm or 1-4 cm in vertical length;
b) the substrate includes a cone tip configured to penetrate a soil;
c) the substrate is rectangular in shape;
d) the substrate comprises a resin material having a post-cured tensile strength of greater than 40 MPa, or greater than 60 MPa;
e) the substrate comprises a resin material having a post-cured notched izod impact strength of greater than 35 J/m
f) the substrate comprises a resin material having a post-cured heat deflection temperature at 1.8 MPa of between 50-55° C.;
g) the substrate comprises a resin material having a post-cured heat deflection temperature at 0.45 MPa of between 60-65° C.;
h) the substrate comprises a resin material having a post-cured thermal expansion of between 85-95 μm/m/° C. at 0-150° C.; or
i) a combination of a)-h).

24. The method of claim 16, wherein:
a) the two grooves on the front surface are between 0.5-10 mm or 1-2 mm in diameter;
b) the two grooves on the front surface are between 0.4-1 mm in depth;
c) the active region is spherical in shape;
d) the active region is between 2.5-3.5 mm in radius;
e) the active region is between 0.4-0.7 mm in depth;
f) a combination of a)-e).
